SEQUENCE LISTING (17 sequences)

SEQ ID NO: 1 - DNA, 1482 bp, Anaerobutyricum soehngenii
  source 1..1482: mol_type = unassigned DNA
tgatcctggctcaggatgaacgctggcggcgtgcctaacacatgcaagtcgaacgaagcaccttttaagattcttcggatgattgatcggtgactgagtggcggacgggtgagtaacgcgtgggtaacctgccctgtacagggggataacagttggaaacggctgctaataccgcataagcgcacgagaggacatcctcttgtgtgaaaaactccggtggtacaggatgggcccgcgtctgattagctggttggcagggtaacggcctaccaaggcgacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgcatagctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatcctaggtgtcggggccgtataggcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccactccgtaatgggagtcttccttcgggacagaagagacaggtggtgcatggttgtccgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacaaagtgaggcaaacctgcgagggggagcaaaccacaaaaaggccgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagccagtgacccaaccttttggagggarctgtcgaaggtggagccggtaactggggtgaagtcgtaacaaggg

SEQ ID NO: 2 - DNA, 1479 bp, Anaerobutyricum hallii
  misc_feature 617: note = n is a, c, g, or t
  misc_feature 955: note = n is a, c, g, or t
  misc_feature 1087: note = n is a, c, g, or t
  misc_feature 1098: note = n is a, c, g, or t
  misc_feature 1184..1185: note = n is a, c, g, or t
  misc_feature 1227: note = n is a, c, g, or t
  misc_feature 1259: note = n is a, c, g, or t
  misc_feature 1269: note = n is a, c, g, or t
  misc_feature 1374..1375: note = n is a, c, g, or t
  misc_feature 1391: note = n is a, c, g, or t
  source 1..1479: mol_type = unassigned DNA
tttatttgagagtttgatcctggctcaggatgaacgctggcggcgtgcctaacacatgcaagtcgaacgaagcaccttaccwgattcttcggatgaaagwytggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggngctgcatttgaaactgcwyrgctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggctycggtgccgccgctaacgcagtaagtattccacctggggagtacgttcgcaagaatgaaactcaaaggaattgacggggacccgcacaagcggtggagcatgtggtttaattcgaagnaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccncaacgagcgcnacccctatcttcagtagccagcaggtaaggctgggcactctggagagactgccagggataacctggaggaaggtggggacgacgtnnaatcatcatgccccttatgatctgggcgacacacgtgctacnatggcggtcacagagtgaggcgaaccygcganggggagcaanccacaaaaaggccgtcccagttcggactgtagtctgcaacccgactacacgaagctggaatcgctagtaatcgcgaatcagaatgtcgcggtgaatacgttcccnngtcttgtacacaccgnccgtcacaccatgggagtcggaaatgcccgaagccagtgacccaacctttatggagggagctgtcgaaggtggagccggtaactgggg

SEQ ID NO: 3 - DNA, 1529 bp, Bifidobacterium animalis subspecies lactis
  source 1..1529: mol_type = unassigned DNA
agtttgatcatggctcaggatgaacgctggcggcgtgcttaacacatgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacccttgtggggggagccgtctaaggtgagactcgtgattgggactaagtcgtaacaaggtagccgtaccggaaggtgcggctggatcacctcctta

SEQ ID NO: 4 - DNA, 1515 bp, Bifidobacterium infantis
  source 1..1515: mol_type = unassigned DNA
tttgatcatggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctcctta

SEQ ID NO: 5 - DNA, 1476 bp, Bifidobacterium longum
  misc_feature 206: note = n is a, c, g, or t
  misc_feature 213: note = n is a, c, g, or t
  misc_feature 256: note = n is a, c, g, or t
  misc_feature 268..269: note = n is a, c, g, or t
  misc_feature 336: note = n is a, c, g, or t
  misc_feature 526: note = n is a, c, g, or t
  misc_feature 820: note = n is a, c, g, or t
  misc_feature 914: note = n is a, c, g, or t
  misc_feature 949: note = n is a, c, g, or t
  misc_feature 1006: note = n is a, c, g, or t
  misc_feature 1033: note = n is a, c, g, or t
  misc_feature 1126: note = n is a, c, g, or t
  misc_feature 1137..1138: note = n is a, c, g, or t
  misc_feature 1275: note = n is a, c, g, or t
  misc_feature 1368: note = n is a, c, g, or t
  misc_feature 1394: note = n is a, c, g, or t
  misc_feature 1443: note = n is a, c, g, or t
  source 1..1476: mol_type = unassigned DNA
ttttgtggagggttcgattctggctcaggatgaacgctggcggcgtgcttaacacatgcaagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctggngaaagcntttcgcggtatgggatggggtcgcgtcctatcagcttgacggnggggtaacggcnnaccgtggcttcgacgggtagccggcctgagagggcgaccggccacattgggactgagatacggcccngactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcnagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggccngttccacgggttccgtgtcggagctaacgcgttaagcatcccgcctggggagtacggccgcaaggctaaaactcaaagaaattgacgggggccngcacaagcggcggagcatgcggattaattcgatgnaacgcgaagaaccttacctgggcttgacatgttcccgacggtcgtagagatacggcntcccttcggggcgggttcacaggtggngcatggtcgtcgtcagctcgtgtcgtgagatgttgggttaagtcccgcaacgagcgcaaccctcgccccgtgttgccagcggattatgccggnaactcacgggnnaccgccggggttaactcggaggaaggtggggatgacgtcagatcatcatgccccttacgtccagggcttcacgcatgctacaatggccggtacaacgggatgcgacgcggcgacgcggagcggatccctgaaaaccngtctcagttcggatcgcagtctgcaactcgactgcgtgaaggcggagtcgctagtaatcgcgaatcagcaacgtcgcggtgaatgcgttcccnggccttgtacacaccgcccgtcaagncatgaaagtgggcagcacccgaagccggtggcctaaccccttgtggganggagccgtctaaggtgaggctcgtgattgggac

SEQ ID NO: 6 - DNA, 1520 bp, Bifidobacterium breve
  source 1..1520: mol_type = unassigned DNA
ttcgattctggctcaggatgaacgctggcggcgtgcttaacacatgcaagtcgaacgggatccatcgggctttgcttggtggtgagagtggcgaacgggtgagtaatgcgtgaccgacctgccccatgcaccggaatagctcctggaaacgggtggtaatgccggatgctccatcacaccgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacagtgcgagctggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgcgggagggagccgtctaaggtgaggctcgtgattgggactaagtcgtaacaaggtagccgtaccggaaggtgcggctggatcacctcctta

SEQ ID NO: 7 - DNA, 1533 bp, Bifidobacterium thermophilum
  source 1..1533: mol_type = unassigned DNA
agagtttgatcatggctcaggatgaacgctggcggcgtgcttaacacatgcaagtcgaacgggatcctgcgggctttgcctgcgggtgagagtggcgaacgggtgagtaatgcgtgaccaacctgccccatgctccggaatagctcctggaaacgggtggtaatgccggatgttcccgcgccccgcatggggtgcggggaaaagcttttgcggcgtgggatggggtcgcgtcctatcagcttgttggcggggtgagggcccaccaaggcttcgacgggtagccggcctgagaaggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgtttgggagcaagcccttcggggtgagtgtacctttcgaataagcaccggctaaatacgtgccagcagccgcggtaataagtagggtgcgagcgttatccggatttattgggcgtaaagggcttgtaggcggtttgtcgcgtccggtgtgaaagtccatcgcctaacggtggatttgcgccgggtacgggcgggctggagtgcggtaggggagactggaattcccggtgtaacggtggaatgtgtagatatcgggaagaacaccaatggcgaaggcaggtctttgggccgttactgacgctgaggagcgaaagcgtggggagcgaacaggattagataccctggtagtccacgccgtaaacggtggatgctggatgtggggcccttccacgggtcccgtgtcggggccaacgcgttaagcatcccgcctggggagtacggccgcaaggctaaaactcaaagaaattgacgggggcccgcacaagcggcggagcatgcggattaattcgatgcaacgcgaaaaaccttacctgggcttgacatgttcccgacgacggcagagatgtcgtttcccttcggggcgggttcacaggtggtgcatggtcgtcgtcagctcgtgtcgtgagatgttgggtcaagtcccgcaacgagcgcaaccctcgccccgtgttgccagcgcgtcttggcgggaactcaccggggaccgccggggtttacccggaggaaggtggggatgacgtcagatcatcatgccccttacgtccagggcttcacggcatgctacaatggccgggtacaggcggggatgcagacatggtgacatggagcgggatccctgaaaaccggtctcagttcgggatcggagcgtgcaacccggctcggtgaaggcggagtcggctaagtaatcgcggatcagcaacgccgcggtgaatgcgttcccgggccttgtacacaccgcccgtcaagtcatgaaagtgggcagcacccgaagccggtggcctgaccagtattgctggggggagccgtctaaggtgaggctcgcgattgggagtaagtcgtaacaaggtagccgtaccggaaggtgcggctggatcacctcctt

SEQ ID NO: 8 - DNA, 1532 bp, Bifdobacterium bifidum
  misc_feature 177: note = n is a, c, g, or t
  misc_feature 237: note = n is a, c, g, or t
  misc_feature 525..528: note = n is a, c, g, or t
  misc_feature 727: note = n is a, c, g, or t
  misc_feature 747: note = n is a, c, g, or t
  misc_feature 916: note = n is a, c, g, or t
  misc_feature 951: note = n is a, c, g, or t
  misc_feature 1139..1140: note = n is a, c, g, or t
  misc_feature 1156: note = n is a, c, g, or t
  misc_feature 1471: note = n is a, c, g, or t
  misc_feature 1483..1484: note = n is a, c, g, or t
  misc_feature 1493..1498: note = n is a, c, g, or t
  misc_feature 1509..1517: note = n is a, c, g, or t
  source 1..1532: mol_type = unassigned DNA
tttttgtggagggttcgattctggctcaggatgaacgctggcggcgtgcttaacacatgcaagtcgaacgggatccatcaagcttgcttggtggtgagagtggcgaacgggtgagtaatgcgtgaccgacctgccccatgctccggaatagctcctggaaacgggtggtaatgccgnatgttccacatgatcgcatgtgattgtgggaaagattctatcggcgtgggatggggtcgngtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggnnnnagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggcngtcactgacgctgaggagcnaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggccngcacaagcggcggagcatgcggattaattcgaacnaacgcgaagaaccttacctgggcttgacatgttcccgacgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggnnaccgccggggttaacncggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggccttgtacacaccgcccgtcaagtcatgaaagtgggcagcacccgaagccggtggcctaaccccttgtgggatggagccgtctaaggtgaggctcgtgnttgggactaagnngtaacaagnnnnnngtaccggaagnnnnnnnnngatcacctcctttct

SEQ ID NO: 9 - DNA, 1477 bp, Bifidobacterium adolescentis
  misc_feature 1..4: note = n is a, c, g, or t
  misc_feature 34: note = n is a, c, g, or t
  misc_feature 41: note = n is a, c, g, or t
  misc_feature 81: note = n is a, c, g, or t
  misc_feature 273: note = n is a, c, g, or t
  misc_feature 290: note = n is a, c, g, or t
  misc_feature 339: note = n is a, c, g, or t
  misc_feature 365: note = n is a, c, g, or t
  misc_feature 513: note = n is a, c, g, or t
  misc_feature 532: note = n is a, c, g, or t
  misc_feature 615: note = n is a, c, g, or t
  misc_feature 636: note = n is a, c, g, or t
  misc_feature 653: note = n is a, c, g, or t
  misc_feature 667: note = n is a, c, g, or t
  misc_feature 731: note = n is a, c, g, or t
  misc_feature 734: note = n is a, c, g, or t
  misc_feature 916..917: note = n is a, c, g, or t
  misc_feature 920..921: note = n is a, c, g, or t
  misc_feature 932: note = n is a, c, g, or t
  misc_feature 954..955: note = n is a, c, g, or t
  misc_feature 1011..1013: note = n is a, c, g, or t
  misc_feature 1023: note = n is a, c, g, or t
  misc_feature 1028: note = n is a, c, g, or t
  misc_feature 1039: note = n is a, c, g, or t
  misc_feature 1132: note = n is a, c, g, or t
  misc_feature 1143: note = n is a, c, g, or t
  misc_feature 1175: note = n is a, c, g, or t
  misc_feature 1283: note = n is a, c, g, or t
  misc_feature 1362: note = n is a, c, g, or t
  misc_feature 1435: note = n is a, c, g, or t
  misc_feature 1466: note = n is a, c, g, or t
  misc_feature 1475: note = n is a, c, g, or t
  source 1..1477: mol_type = unassigned DNA
nnnnttgtggagggttcgattctggctcaggatnaacgctngcggcgtgcttaacacatgcaagtcgaacgggatcggctngagcttgctccggctgtgagagtggcgaacgggtgagtaatgcgtgaccgacctgccccatacaccggaatagctcctggaaacgggtggtaatgccggatgctccagttggatgcatgtccttctgggaaagattctatcggtatgggatggggtcgcgtcctatcagcttgatggcggggtaacggcccnccatggcttcgacgggnagccggcctgagagggcgaccggccacattgggactgagatacggcccngactcctacgggaggcagcagtgggnaatattgcacaatgggcgcaagcctaatgcagcgacgccgcgtgcgggatgacggccttcgggttgtaaaccgcttttgactgggagcaagccttcggggtgagtgtacctttcgaataagcaccggctaactacgtgccagcagccncggtaatacgtagggtgcnagcgttatccggaattattgggcgtaaagggctcgtaggcggttcgtcgcgtccggtgtgaaagtccatcgcttaacggtggntccgcgccgggtacgggcggncttgagtgcggtagggnagactggaattccnggtgtaacggtggaatgtgtagatatcgggaagaacaccaatggcgaaggcaggtctctgggcngtnactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggnnccnncacaagcggcngagcatgcggattaattcgatnnaacgcgaagaaccttacctgggcttgacatgttcccgacaggccccagagatgggnnntccttcgggncgggntcacaggtggngcatggtcgtcgtcagctcgtgtcgtgagatgttgggttaagtcccgcaacgagcgcaaccctcgccctgtgttgccagcacgtcgtggtggnaactcacgggngaccgccggggtcaactcggaggaaggtgggnatgacgtcagatcatcatgccccttacgtccagggcttcacgcatgctacaatggccggtacaacgggatgcgacctcgtgagggggagcggatcccttaaaaccggnctcagttcggattggagtctgcaacccgactccatgaaggcggagtcgctagtaatcgcggatcagcaacgccgcggtnaatgcgttcccgggccttgtacacaccgcccgtcaagccatgaaagtgggtagcacccgaagccggtggcccnacctttttggggggagccgtctaaggtgagnctcgtgatngg

SEQ ID NO: 10 - DNA, 1478 bp, Bifodbacterium catenulatum
  misc_feature 1..3: note = n is a, c, g, or t
  misc_feature 13: note = n is a, c, g, or t
  misc_feature 96: note = n is a, c, g, or t
  misc_feature 113..114: note = n is a, c, g, or t
  misc_feature 129: note = n is a, c, g, or t
  misc_feature 137..138: note = n is a, c, g, or t
  misc_feature 208: note = n is a, c, g, or t
  misc_feature 238: note = n is a, c, g, or t
  misc_feature 269..271: note = n is a, c, g, or t
  misc_feature 280: note = n is a, c, g, or t
  misc_feature 336..337: note = n is a, c, g, or t
  misc_feature 363: note = n is a, c, g, or t
  misc_feature 389: note = n is a, c, g, or t
  misc_feature 402..404: note = n is a, c, g, or t
  misc_feature 412: note = n is a, c, g, or t
  misc_feature 422: note = n is a, c, g, or t
  misc_feature 436: note = n is a, c, g, or t
  misc_feature 438: note = n is a, c, g, or t
  misc_feature 473: note = n is a, c, g, or t
  misc_feature 529: note = n is a, c, g, or t
  misc_feature 651: note = n is a, c, g, or t
  misc_feature 716: note = n is a, c, g, or t
  misc_feature 728: note = n is a, c, g, or t
  misc_feature 730..731: note = n is a, c, g, or t
  misc_feature 822..823: note = n is a, c, g, or t
  misc_feature 865: note = n is a, c, g, or t
  misc_feature 874: note = n is a, c, g, or t
  misc_feature 877: note = n is a, c, g, or t
  misc_feature 882: note = n is a, c, g, or t
  misc_feature 890..894: note = n is a, c, g, or t
  misc_feature 896: note = n is a, c, g, or t
  misc_feature 909: note = n is a, c, g, or t
  misc_feature 917: note = n is a, c, g, or t
  misc_feature 928: note = n is a, c, g, or t
  misc_feature 943: note = n is a, c, g, or t
  misc_feature 950..952: note = n is a, c, g, or t
  misc_feature 1008: note = n is a, c, g, or t
  misc_feature 1025..1026: note = n is a, c, g, or t
  misc_feature 1036: note = n is a, c, g, or t
  misc_feature 1051: note = n is a, c, g, or t
  misc_feature 1083: note = n is a, c, g, or t
  misc_feature 1126: note = n is a, c, g, or t
  misc_feature 1129: note = n is a, c, g, or t
  misc_feature 1139..1140: note = n is a, c, g, or t
  misc_feature 1279: note = n is a, c, g, or t
  misc_feature 1388: note = n is a, c, g, or t
  misc_feature 1396: note = n is a, c, g, or t
  misc_feature 1431: note = n is a, c, g, or t
  misc_feature 1436: note = n is a, c, g, or t
  source 1..1478: mol_type = unassigned DNA
nnnttttgtgagnggttcgattctggctcaggatgaacgctggcggcgtgcttaacacatgcaagtcgaacgggatcaggcagcttgctgcctggngagagtggcgaacgggnnagtaatgcgtgaccnacctgccnnatacaccggaatagctcctggaaacgggtggtaatgccggatgctccgactcctcgcatggggtgtcggnaaagatttcatcggtatgggatggggtcgngtcctatcaggtagtcggcggggtaacggcnnnccgagcctncgacgggtagccggcctgagagggcgaccggccacattgggactgagatacggccnngactcctacgggaggcagcagtgggncatattgcacaatgggcgcaagcctnatgcagcgacgcnnngtgcgggntgacggcctncgggttgtaaaccncntttgatcgggagcaagccttcgggtgagtgtaccnttcgaataagcaccggctaactacgtgccagcagccgcggtaatacgtagggtgcnagcgttatccggaattattgggcgtaaagggctcgtaggcggttcgtcgcgtccggtgtgaaagtccatcgcttaacggtggatctgcgccgggtacgggcgggctggagtgcggtaggggngactggaattcccggtgtaacggtggaatgtgtagatatcgggaagaacaccaatggcgaaggcnggtctctgggcngnnactgacgctgaggagcgaaagcgtggggagcgaacaggattagataccctggtagtccacgccgtaaacggtggatgctggatgtggggcnngttccacgggttccgtgtcggagctaacgcgttaagcatccngcctggggngtncggcngcaaggcnnnnncncaaagaaattgangggggccngcacaagcggnggagcatgcggattnattcgannnaacgcgaagaaccttacctgggcttgacatgttcccgacagccgtagagatacggnctcccttcggggcgggnncacaggtggngcatggtcgtcgtcngctcgtgtcgtgagatgttgggttaagtcccncaacgagcgcaaccctcgccctgtgttgccgacacgtcatgtnggnactcacgggnnaccgccggggtcaactcggaggaaggtggggatgacgtcagatcatcatgccccttacgtccagggcttcacgcatgctacaatggccggtacaacgggatgcgacatggcgacatggagcggatccctgaaaaccggnctcagttcggattggagtctgcaacccgactccatgaaggcggagtcgctagtaatcgcggatcagcaacgccgcggtgaatgcgttcccgggccttgtacacaccgcncgtcaagncatgaaagtgggtagcacccgaagccggtggcctnacccnttgtgggatggagccgtctaaggtgagactcgtgattgggac

SEQ ID NO: 11 - DNA, 1519 bp, Bifdobacterium pseudocatenulatum
  source 1..1519: mol_type = unassigned DNA
gtttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttgtggatggagccgtctaaggtgagactcgtgattgggactaagtcgtaacaaggtagccgtaccggaaggtgcggctggatcacctcctta

SEQ ID NO: 12 - DNA, 1433 bp, Akkermansia muciniphila
  source 1..1433: mol_type = unassigned DNA
aacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagtaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagctggtcgcacccgaagtatctgaagccaaccgcaaggaggcagggtcctaaggtgagactggtaactgggatg

SEQ ID NO: 13 - DNA, 1438 bp, Akkermansia glycanipila
  source 1..1438: mol_type = unassigned DNA
aacgaacgctggcggcgtggataagacatgcaagtcgaacggagaagcaatagcttgctaatgcttcttagtggcgcacgggtgagtaacacgtgagcaacctgccttcgagacgggaatagccctgggaaaccgggattaatgcccgatagactcgcaagagtaaacgcagcaatgcgcttgaagaggggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaagaaggtcttcggattgtaaacccctgtcatgtgggagcaaggcgcaagcttgatagtaccacaagaggaagagacggctaactctgtgccagcagccgcggtaatacagaggtctcaagcgttgttcggaatcactgggcgtaaagggtacgtaggctgcatcataagtcgggcgtgaaaggcaggggctcaacccctggagtgcgcttgatactgtgatgctagagtcatggagggggaaccggaactctcggtgtagcagtgaaatgcgtagatatcgagaagaacactcgtggcgaaggcgggttcctggacatgtactgacgctgaggtacgaaggctaggggagcgaaagggattagatacccctgtagtcctagcagtaaacggtgcacgcttggtgtgtggggaatcgaccccccacgtgccggagcaaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtgatgaacaacatgtgaaagcatgtgacacctcggtggcgtcacacaggtgctgcatggccgtcgtcagctcgtgtcgtgagatgtttggttaagtccagcaacgagcgcaacccctgttgccagttaccagcacgttatggtggggactctggcgagactgcccagatcaactgggaggaaggtggggacgacgtcaggtcagtatggcccttatgcccagggctgcacacgtactacaatgcccagtacagagggtaccgaacccgcgagggggaggcaatccatgaaaactgggcccagttcggattgtaggctgcaactcgcctacatgaagatggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaa

SEQ ID NO: 14 - DNA, 1478 bp, Lactobacillus acidophilus
  source 1..1478: mol_type = unassigned DNA
tcctggctcaggacgaacgctggcggcgtgcctaatacatgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggc

SEQ ID NO: 15 - DNA, 671 bp, Lactobacillus casei
  source 1..671: mol_type = unassigned DNA
gttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatctttttgatcactgagagatcaggtttccccttcgggggcaaaatgacaggtggtgcatgttgtcgtcagctcgtgtcgtgagatgttgggttaagtcccgcaacgagcgcta

SEQ ID NO: 16 - DNA, 1535 bp, Lactobacillus reuteri
  source 1..1535: mol_type = unassigned DNA
agagtttgatcctggctcaggatgaacgccggcagtgtgcctaatacatgcaagtcgtacgcactggcccaactaattgatggtgcttgctgaattgacgatggatcaccagtgagtggcggacgggtgagtaacacgtaggtaacctgccccggagcggggaataacatttggaaacagatgctaataccgcataacaacaaaagccgcatggtttttctggaaagatggctttggctatcactctgggatggacctgcggtgcatttagctagttggtaaggtaacggcttacccaaggcgatgatgcatagccgagttgagagactgatcggccacaatgggaactgagacacggtccataacttctacgggaggcagcagtagggaatcttccacaatgggcgcaagctgatggagcaacaccgcgttattaagaaagggtttcggccgcttaaactctgttgttggagaagaacgtgcgttagagtaactgttacgcagtgacggtatccaaccagaaagtcacggctaactacgtgccagcagccgcggtaatacgtaggtggcaagcgttatccggatttattgggcgtaaagcgagcgcaggcggttgcttaggtctgatgtggaaactcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgcctgttctaacgcattaatgcactccgcctggggagtacgaccgcaaggttgaaactcaaaggaattgacgggggcccgcacaagcggtgaagcatgtggtttaattcgaagctacgcgaagaaccttaccaggtcttgacatcttgcgctaaccttagagataaggcgttcccttcggggacgttaatgacaggtggtgcatggtcgtcgtcagctcgtgtcgtgagatgttgggttaagtcccgcaacgagcgcaacccttgttactagttgccagcattaagttggggactctagtgagactgccggtgacaaaccggaggaaggtggggacgacgtcagatcatcatgccccttatgaccctgggctacacacgtgctacaatggacggtacaacgagtcgcaaactcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagttcggtggcctaacctttatggacgggtaccctaaggcgggacagatgatctggggtgaagtcgtaacaaggta

SEQ ID NO: 17 - DNA, 1521 bp, Lactobacillus rhamnosus
  misc_feature 407: note = n is a, c, g, or t
  misc_feature 655: note = n is a, c, g, or t
  misc_feature 1476: note = n is a, c, g, or t
  source 1..1521: mol_type = unassigned DNA
grtsaacgctsgcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactctacgggaggcagcagtagggaatcttccacaatggacgcaagtctgatggagcaacgccgcgtgagtnaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtncagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggncaaatgattagggtgaagtcgtaacaaggtagccgtaggagaacc

(19) United States
(12) Patent Application Publication
(10) Pub. No.: US 2025/0025517 A1
(43) Pub. Date: Jan. 23, 2025

(54) PREVENTION OR TREATMENT OF HEPATIC STEATOSIS

(71) Applicants: CAELUS PHARMACEUTICALS B.V., Zegveld (NL); STICHTING AMSTERDAM UMC, Amsterdam (NL)

(72) Inventors: A.G. Holleboom, Amsterdam (NL); Max Nieuwdorp, Amsterdam (NL); Willem Meindert De Vos, Amsterdam (NL)

(21) Appl. No.: 18/713,999

(22) PCT Filed: Nov. 30, 2022
(86) PCT No.: PCT/EP2022/083885

§ 371 Date: May 28, 2024

(30) Foreign Application Priority Data
Dec. 3, 2021 (NL) 2030011

(51) Int. Cl. A61K 35/745; A61K 35/747; A61P 1/16; A61P 29/00

(52) U.S. Cl. CPC A61K 35/745; A61K 35/747; A61P 1/16; A61P 29/00

(57) ABSTRACT

This disclosure is concerned with Anaerobutyricum soehngenii or relative thereof for use in preventing and/or treating hepatic steatosis, particularly nonalcoholic fatty liver disease (NAFLD) and/or nonalcoholic steatohepatitis (NASH), wherein the use is for increasing bile acid plasma level for reducing liver inflammation and/or for reducing hepatic necroinflammatory activity score. The Anaerobutyricum soehngenii or relative thereof may be combined with at least one Bifidobacterium species, preferably Bifidobacterium animalis subspecies lactis or relative thereof and/or Bifidobacterium breve or relative thereof. In addition or alternatively, the Anaerobutyricum soehngenii or relative thereof may be combined with at least one Akkermansia species, preferably Akkermansia muciniphila or relative thereof. In addition or alternatively, the Anaerobutyricum soehngenii or relative thereof may be combined with at least one Lactobacillus species, preferably Lactobacillus acidophilus or relative thereof, Lactobacillus casei or relative thereof and/or Lactobacillus reuteri or relative thereof.

### CROSS-REFERENCE TO RELATED APPLICATIONS

This application is a national phase entry under 35 U.S.C. § 371 of International Patent Application PCT/EP2022/083885, filed Nov. 30, 2022, designating the United States of America and published as International Patent Publication WO 2023/099579 A1 on Jun. 8, 2023, which claims the benefit under Article 8 of the Patent Cooperation Treaty of Netherlands Patent Application Serial No. 2030011, filed Dec. 3, 2021.

### STATEMENT REGARDING ELECTRONIC FILING OF A SEQUENCE LISTING

Pursuant to 37 C.F.R. § 1.834, a Sequence Listing XML file entitled “6WD5763.XML Sequence listing ST26-NOV2022.xml,” 88.0 kilobytes in size, generated May 19, 2024, has been submitted via EFS-Web and is provided in lieu of a paper copy. This Sequence Listing is hereby incorporated by reference into the specification for its disclosures.

### TECHNICAL FIELD

The present disclosure relates to the field of preventing and/or treating hepatic steatosis.

### BACKGROUND

Non-alcoholic fatty liver disease (NAFLD) is recognized as the most prevalent chronic liver disease worldwide, and its spectrum ranges from simple steatosis (non-alcoholic fatty liver) to non-alcoholic steatohepatitis (NASH), NASH-fibrosis, cirrhosis and hepatocellular carcinoma. The current estimated global prevalence of NAFLD is 25%-30% in the general population, and up to 80% in individuals with metabolic syndrome and Type 2 Diabetes mellitus. By definition, excessive alcohol use precludes a diagnosis of NAFLD.

NAFLD refers to a spectrum of disease in which excess fat accumulates in the liver in patients who drink little or no alcohol. The most common form of NAFLD is called non-alcoholic fatty liver (NAFLD). As the occurrence and progression of NAFLD are strongly driven by insulin resistance, multiple therapeutic strategies in clinical development for NAFLD aim at reducing insulin resistance.

The gut microbiota has been linked to the development and prevalence of NAFLD and NASH. Disease occurrence is significantly lower in individuals taking a plant-based, low-animal-protein diet, which is thought to be mediated by gut microbiota. Hence, Witjes at al. (Hepatology Communications, Vol. 4, no. 11, 2020) propose transplantation of fecal microbiota from lean vegan donors as a potential treatment.

However, there is a need in the art for new and improved interventions in the prevention and treatment of NAFLD and NASH.

It is an object of the present disclosure, amongst other objects, to address the above need in the art to provide a new and/or improved strategy for preventing and/or treating NAFLD and NASH.

### BRIEF SUMMARY

Surprisingly, it was found that administration of Anaerobutyricum soehngenii, or relative thereof, to subjects having hepatic steatosis, increases bile acid plasma levels, which reduces liver inflammation. Accordingly, administration of Anaerobutyricum soehngenii, or relative thereof may be applied in a strategy for prevention and/or treatment of hepatic steatosis.

In addition, it was found that combining Anaerobutyricum soehngenii, or relative thereof, with a Bifidobacterium species, an Akkermansia species and/or a Lactobacillus species provides a synergistic therapeutic effect in the prevention or treatment of hepatic steatosis, in particular in Nonalcoholic fatty liver disease (NAFLD), and/or nonalcoholic steatohepatitis (NASH).

The present disclosure provides a new and improved strategy for preventing and/or treating hepatic steatosis, NAFLD, and/or NASH.

### BRIEF DESCRIPTION OF THE DRAWINGS

FIG. 1: SCFA production in the absence or presence of Bifidobacterium animalis subsp lactis BLC1.

FIG. 2: SCFA produced in absence or presence of L.rhamnosus GG on fucose (25 mM) in YCFA medium.

FIG. 3: Histological evaluation of the mice. Panels A-D: Inflammation grade, fibrosis grade, NAS score or global NASH score of the mice, and Panel E: CRN classification.

### DETAILED DESCRIPTION

The present disclosure relates to Anaerobutyricum soehngenii or relative thereof having a 16S rRNA gene sequence with at least 70, 80, 85, 90, 95, 96, 97, 98, 99, 99.5, 99.9, 100% sequence identity with SEQ ID NO:1 and/or SEQ ID NO:2, particularly for use in preventing and/or treating hepatic steatosis, and/or for increasing production of propionic acid/propionate and/or butyric acid/butyrate or a derivative thereof in the intestine.

In accordance with the foregoing, the present disclosure relates to a method for preventing and/or treating hepatic steatosis, e.g., in a subject in need thereof, involving administration, e.g., to the subject, of the Anaerobutyricum soehngenii or relative thereof.

Hepatic steatosis is a condition where excess fat builds up in the liver. There are two stages of fatty liver disease: non-alcoholic fatty liver disease (NAFLD) and alcoholic liver disease. NAFLD is made up of simple fatty liver and non-alcoholic steatohepatitis (NASH).

In the present disclosure, the hepatic steatosis may in a particular be chosen from Nonalcoholic fatty liver disease (NAFLD) and/or nonalcoholic steatohepatitis (NASH).

The term “Nonalcoholic fatty liver disease” (NAFLD) refers to a group of conditions where there is accumulation of excess fat in the liver of people who drink little or no alcohol. The most common stage of NAFLD is called fatty liver. NAFLD is strongly associated with insulin resistance and type 2 diabetes mellitus, therefore, treatments of NAFLD may aim at lowering insulin resistance.

The term “Nonalcoholic steatohepatitis” (NASH) refers to liver inflammation and damage caused by a buildup of fat in the liver. NASH is associated with a markedly increased risk of developing cirrhosis and hepatocellular carcinoma as well as other diseases not directly associated with liver damage, including increased risk of cardiovascular disease. An association between insulin resistance and the development of NASH (/NAFLD) is well-known, and strategies to lower insulin resistance may decrease disease progression or symptoms in NASH (/NAFLD).

The use according to the disclosure can increase plasma levels of bile acids, in particular primary bile acids (cholic acid and chenodeoxycholic acid) and/or secondary bile acids (deoxycholic acid and lithocholic acid). This, in turn, reduces liver inflammation (e.g., as determined by (sum of) lobular inflammation score 0-3, microgranulomas score 0-1, large lipogranulomas score 0-1, and/or portal inflammation score 0-1 as shown below); or as determined by necroinflammatory activity score (NAS). Hence, the use according to the disclosure can reduce liver inflammation (e.g., as determined by (sum of) lobular inflammation score 0-3, microgranulomas score 0-1, large lipogranulomas score 0-1, and/or portal inflammation score 0-1 as shown below); or as determined by necroinflammatory activity score.

An increase in bile acid plasma level as part of the current disclosure is preferably indicated by one or more of the following methods: thin-layer chromatography, gas chromatography, high-performance liquid chromatography (HPLC), liquid chromatography-mass spectrometry (LC-MS), gas chromatography-mass spectrometry (GC-MS) supercritical fluid chromatography and capillary electrophoresis, immunoassays, and bioluminescence assays.

In a particularly preferred embodiment, the use according to the present disclosure is for reducing hepatic necroinflammatory activity score.

The term “hepatic necroinflammatory activity score” is interchangeable with the terms “NAFLD score” and/or “NASH score.”

To determine the hepatic necroinflammatory activity score, the NASH Clinical Research Network (NASH-CRN) classification may be used as described by Kleiner et al., Volume 41, Issue 6 June 2005), e.g., with use of hematoxylin and eosin-stained slides for steatosis, inflammation, and ballooning, and with a sirius red-stained slide for evaluation of fibrosis. The score preferably is the unweighted sum of steatosis grade (0-3), lobular inflammation (0-3), and hepatocellular ballooning (0-2), see below:

| Steatosis | | |
|---|---|---|
| Grade | Low- to medium-power evaluation of | |
| | parenchymal involvement by steatosis | |
| | <5% | 0 |
| | 5%-33% | 1 |
| | >33%-66% | 2 |
| | >66% | 3 |
| Location | Predominant distribution pattern | |
| | Zone 3 | 0 |
| | Zone 1 | 1 |
| | Azonal | 2 |
| | Panacinar | 3 |
| Microvesicular | Contiguous patches | |
| steatosis* | Not present | 0 |
| | Present | 1 |
| Fibrosis | | |
| Stage | | |
| | None | 0 |
| | Perisinusoidal or periportal | 1 |
| | Mild, zone 3, perisinusoidal | 1A |
| | Moderate, zone 3, perisinusoidal | 1B |
| | Portal/periportal | 1C |
| | Perisinusoidal and portal/periportal | 2 |
| | Bridging fibrosis | 3 |
| | Cirrhosis | 4 |
| Inflammation | | |
| Lobular inflammation | Overall assessment of all | |
| | inflammatory foci | |
| | No foci | 0 |
| | <2 foci per 200x field | 1 |
| | 2-4 foci per 200x field | 2 |
| | >4 foci per 200x field | 3 |
| Microgranulomas | Small aggregates of macrophages | |
| | Absent | 0 |
| | Present | 1 |
| Large lipogranulomas | Usually in portal areas or adjacent | |
| | to central veins | |
| | Absent | 0 |
| | Present | 1 |
| Portal inflammation | Assessed from low magnification | |
| | None to minimal | 0 |
| | Greater than minimal | 1 |
| Liver cell injury | | |
| Ballooning* | None | 0 |
| | Few balloon cells | 1 |
| | Many cells/prominent ballooning | 2 |
| Acidophil bodies | | |
| | None to rare† | 0 |
| | Many | 1 |
| Pigmented macrophages | | |
| | None to rare† | 0 |
| | Many | 1 |
| Megamitochondria* | | |
| | None to rare† | 0 |
| | Many | 1 |
| Other findings | | |
| Mallory's hyaline | Visible on routine stains | |
| | None to raret | 0 |
| | Many | 1 |
| Glycogenated nuclei | Contiguous patches | |
| | None to rare† | 0 |
| | Many | 1 |
| Diagnostic classification‡ | | |
| Not steatohepatitis | | 0 |
| Possible/borderline | | 1 |
| Definite steatohepatitis | | 2 |

*Ballooning classification: few indicates rare but definite ballooned hepatocytes as well as case that are diagnostically borderline.
†The “None to rare” category is meant to alleviate the need for time-consuming searches for rare examples or deliberation over diagnostically borderline changes. If the feature is identified after a reasonable search, it should be coded as “many.”
‡Diagnostic classification may not be available on adult biopsy observations.

The use according to the disclosure can also decrease:
steatosis grade score, particularly as defined above (score 1, 2, 3); and/or
fibrosis stage score, particularly as defined above (score 1, 1A, 1B, 1C, 2, 3, or 4).

The Anaerobutyricum soehngenii or relative thereof according to the present disclosure is preferably chosen from Anaerobutyricum species or Eubacterium species, preferably Anaerobutyricum soehngenii (e.g., DSM17630/KCTC15707) and/or Anaerobutyricum hallii (DSM3353/ATCC27751).

In a study by Shetty et al. (Int. J. Syst. Evol. Microbiol. 2018 December; 68 (12): 3741-3746), the species formerly known as Eubacterium hallii has been reclassified into two groups: Anaerobutyricum hallii and Anaerobutyricum soehngenii. Both Anaerobutyricum soehngenii and/or Anaerobutyricum hallii are considered as an anaerobic Gram-positive, catalase-negative bacterium belonging to the clostridial cluster XIVa (also known as Lachnospiracaea) of the phylum Firmicutes.

Most preferably the at least one Anaerobutyricum species according to the present disclosure is Anaerobutyricum soehngenii (e.g., DSM17630/KCTC15707), or a relative thereof having a 16S rRNA gene sequence with at least 70, 80, 85, 90, 95, 96, 97, 98, 99, 99.5, 99.9, 100% sequence identity with the 16S rDNA sequence of Anaerobutyricum soehngenii (SEQ ID NO: 1). Such cut-off value based on 16S rDNA similarity can define species with similar characteristics and/or functionality.

In addition or alternatively, the Anaerobutyricum species according to the present disclosure is Anaerobutyricum hallii (e.g., DSM3353/ATCC27751), or a relative thereof having a 16S rRNA gene sequence with at least 70, 80, 85, 90, 95, 96, 97, 98, 99, 99.5, 99.9, 100% sequence identity with the 16S rDNA sequence of Anaerobutyricum hallii (SEQ ID NO:2). Such cut-off value based on 16S rDNA similarity can define species with similar characteristics and/or functionality.

Anaerobutyricum soehngenii L2-7 16S IRNA gene sequence
Nucleotide sequence (SEQ ID NO: 1) *
tgatcctggc tcaggatgaa cgctggcggc gtgcctaaca catgcaagtc gaacgaagca
ccttttaaga ttcttcggat gattgatcgg tgactgagtg gcggacgggt gagtaacgcg
tgggtaacct gccctgtaca gggggataac agttggaaac ggctgctaat accgcataag
cgcacgagag gacatcctct tgtgtgaaaa actccggtgg tacaggatgg gcccgcgtct
gattagctgg ttggcagggt aacggcctac caaggcgacg atcagtagcc ggtctgagag
gatgaacggc cacattggaa ctgagacacg gtccaaactc ctacgggagg cagcagtggg
gaatattgca caatggggga aaccctgatg cagcaacgcc gcgtgagtga agaagtattt
cggtatgtaa agctctatca gcagggaaga taatgacggt acctgactaa gaagctccgg
ctaaatacgt gccagcagcc gcggtaatac gtatggagca agcgttatcc ggatttactg
ggtgtaaagg gtgcgtaggt ggcagtgcaa gtcagatgtg aaaggccggg gctcaacccc
ggagctgcat ttgaaactgc atagctagag tacaggagag gcaggcggaa ttcctagtgt
agcggtgaaa tgcgtagata ttaggaggaa caccagtggc gaaggcggcc tgctggactg
ttactgacac tgaggcacga aagcgtgggg agcaaacagg attagatacc ctggtagtcc
acgccgtaaa cgatgaatcc taggtgtcgg ggccgtatag gcttcggtgc cgtcgcaaac
gcagtaagta ttccacctgg ggagtacgtt cgcaagaatg aaactcaaag gaattgacgg
ggacccgcac aagcggtgga gcatgtggtt taattcgaag caacgcgaag aaccttacca
ggtcttgaca tccttctgac cactccgtaa tgggagtctt ccttcgggac agaagagaca
ggtggtgcat ggttgtccgt cagctcgtgt cgtgagatgt tgggttaagt cccgcaacga
gcgcaacccc tatcttcagt agccagcagg taaggctggg cactctggag agactgccag
ggataacctg gaggaaggtg gggacgacgt caaatcatca tgccccttat gatctgggcg
acacacgtgc tacaatggcg gtcacaaagt gaggcaaacc tgcgaggggg agcaaaccac
aaaaaggccg tcccagttcg gactgtagtc tgcaacccga ctacacgaag ctggaatcgc
tagtaatcgc gaatcagaat gtcgcggtga atacgttccc gggtcttgta cacaccgccc
gtcacaccat gggagtcgga aatgcccgaa gccagtgacc caaccttttg gagggarctg
tcgaaggtgg agccggtaac tggggtgaag tcgtaacaag gg
Anaerobutyricum hallii 16S rRNA gene sequence
Nucleotide sequence (SEQ ID NO: 2)*
tttatttgag agtttgatcc tggctcagga tgaacgctgg cggcgtgcct aacacatgca
agtcgaacga agcaccttac cwgattcttc ggatgaaagw ytggtgactg agtggcggac
gggtgagtaa cgcgtgggta acctgccctg tacaggggga taacagctgg aaacggctgc
taataccgca taagcgcacg aggagacatc tccttgtgtg aaaaactccg gtggtacagg
atgggcccgc gtctgattag ctggttggca gggtaacggc ctaccaaggc aacgatcagt
agccggtctg agaggatgaa cggccacatt ggaactgaga cacggtccaa actcctacgg
gaggcagcag tggggaatat tgcacaatgg gggaaaccct gatgcagcaa cgccgcgtga
gtgaagaagt atttcggtat gtaaagctct atcagcaggg aagataatga cggtacctga
ctaagaagct ccggctaaat acgtgccagc agccgcggta atacgtatgg agcaagcgtt
atccggattt actgggtgta aagggtgcgt aggtggcagt gcaagtcaga tgtgaaaggc
cggggctcaa ccccggngct gcatttgaaa ctgcwyrgct agagtacagg agaggcaggc
ggaattccta gtgtagcggt gaaatgcgta gatattagga ggaacaccag tggcgaaggc
ggcctgctgg actgttactg acactgaggc acgaaagcgt ggggagcaaa caggattaga
taccctggta gtccacgccg taaacgatga atactaggtg tcggggccgt ataggctycg
gtgccgccgc taacgcagta agtattccac ctggggagta cgttcgcaag aatgaaactc
aaaggaattg acggggaccc gcacaagcgg tggagcatgt ggtttaattc gaagnaacgc
gaagaacctt accaggtctt gacatccttc tgaccgcacc ttaatcggtg ctttccttcg
ggacagaaga gacaggtggt gcatggttgt cgtcagctcg tgtcgtgaga tgttgggtta
agtcccncaa cgagcgcnac ccctatcttc agtagccagc aggtaaggct gggcactctg
gagagactgc cagggataac ctggaggaag gtggggacga cgtnnaatca tcatgcccct
tatgatctgg gcgacacacg tgctacnatg gcggtcacag agtgaggcga accygcgang
gggagcaanc cacaaaaagg ccgtcccagt tcggactgta gtctgcaacc cgactacacg
aagctggaat cgctagtaat cgcgaatcag aatgtcgcgg tgaatacgtt cccnngtctt
gtacacaccg nccgtcacac catgggagtc ggaaatgccc gaagccagtg acccaacctt
tatggaggga gctgtcgaag gtggagccgg taactgggg
*“n” refers to a, t, c, or g.

In a preferred embodiment, the Anaerobutyricum soehngenii or relative thereof according the disclosure is combined with at least one Bifidobacterium species. It was found that this is a synergistic combination, leading to an unexpected reduction in hepatic necroinflammatory activity score.

The Bifidobacterium species may be administered separately, sequentially or simultaneously with Anaerobutyricum soehngenii or relative thereof. Accordingly, the Bifidobacterium species may be comprised in the same or in a separate composition with respect to Anaerobutyricum soehngenii or relative thereof.

Bifidobacterium is a genus of gram-positive, typically nonmotile, often branched anaerobic bacteria. They are ubiquitous inhabitants of the gastrointestinal tract, vagina and mouth of mammals, including humans. Bifidobacteria are one of the major genera of bacteria that make up the gastrointestinal tract microbiota in mammals. The at least one Bifidobacterium species according to the present disclosure is/are preferably able to assimilate human milk oligosaccharides (HMOs).

The at least one Bifidobacterium species of the present disclosure preferably includes one or more of:
Bifidobacterium animalis sub. lactis, or relative thereof having a 16S rRNA gene with at least 90, 95, 97, 98, 99, 100% sequence identity with the 16S rRNA gene sequence of the type strain of Bifidobacterium animalis sub. lactis (NCBI accession code NR_040867, SEQ ID NO:3);
Bifidobacterium infantis (able to assimilate HMO), or relative thereof having a 16S rRNA gene with at least 90, 95, 97, 98, 99, 100% sequence identity with the 16S rRNA gene sequence of the type strain of Bifidobacterium infantis (NCBI accession code D86184, SEQ ID NO:4);
Bifidobacterium longum (able to assimilate HMO), or relative thereof having a 16S rRNA gene with at least 90, 95, 97, 98, 99, 100% sequence identity with the 16S rRNA gene sequence of the type strain of Bifidobacterium longum (NCBI accession code M58739, SEQ ID NO:5);
Bifidobacterium breve (able to assimilate HMO), or relative thereof having a 16S rRNA gene with at least 90, 95, 97, 98, 99, 100% sequence identity with the 16S rRNA gene sequence of the type strain of Bifidobacterium breve (NCBI accession code AB006658, SEQ ID NO:6);
Bifidobacterium thermophilum, or relative thereof having a 16S rRNA gene with at least 90, 95, 97, 98, 99, 100% sequence identity with the 16S rRNA gene sequence of the type strain of Bifidobacterium thermophilum (NCBI accession code AB016246, SEQ ID NO:7);
Bifdobacterium bifidum, or relative thereof having a 16S rRNA gene with at least 90, 95, 97, 98, 99, 100% sequence identity with the 16S rRNA gene sequence of the type strain of Bifdobacterium bifidum (NCBI accession code M38018, SEQ ID NO: 8);
Bifidobacterium adolescentis, or relative thereof having a 16S rRNA gene with at least 90, 95, 97, 98, 99, 100% sequence identity with the 16S rRNA gene sequence of the type strain of Bifidobacterium adolescentis (NCBI accession code M58729, SEQ ID NO:9);
Bifodbacterium catenulatum or relative thereof having a 16S rRNA gene with at least 90, 95, 97, 98, 99, 100% sequence identity with the 16S rRNA gene sequence of the type strain of Bifodbacterium catemilatum (NCBI accession code M58732, SEQ ID NO:10);
Bifdobacterium pseudocatenulatum or relative thereof having a 16S rRNA gene with at least 90, 95, 97, 98, 99, 100% sequence identity with the 16S rRNA gene sequence of the type strain of Bifdobacterium pseudocatenulatum (NCBI accession code D86187, SEQ ID NO:11).

In a particularly preferred embodiment, the Bifidobacterium species is chosen from:
Bifidobacterium animalis subspecies lactis or relative thereof having a 16S rRNA gene sequence with at least 90, 95, 97, 99, 100% sequence identity with SEQ ID NO:3; and/or
Bifidobacterium breve or relative thereof having a 16S rRNA gene sequence with at least 90, 95, 97, 99, 100% sequence identity with SEQ ID NO:6.

Bifidobacterium animalis subspecies lactis 16S rRNA gene (NCBI/Genbank accession code
NR_040867, SEQ ID NO: 3)

| | |
|---|---|
| 1 | agtttgatca tggctcagga tgaacgctgg cggcgtgctt aacacatgca agtcgaacgg |
| 61 | gatccctggc agcttgctgt cggggtgaga gtggcgaacg ggtgagtaat gcgtgaccaa |
| 121 | cctgccctgt gcaccggaat agctcctgga aacgggtggt aataccggat gctccgctcc |
| 181 | atcgcatggt ggggtgggaa atgcttttgc ggcatgggat ggggtcgcgt cctatcagct |
| 241 | tgttggcggg gtgatggccc accaaggcgt tgacgggtag ccggcctgag agggtgaccg |
| 301 | gccacattgg gactgagata cggcccagac tcctacggga ggcagcagtg gggaatattg |
| 361 | cacaatgggc gcaagcctga tgcagcgacg ccgcgtgcgg gatggaggcc ttcgggttgt |
| 421 | aaaccgcttt tgttcaaggg caaggcacgg tttcggccgt gttgagtgga ttgttcgaat |
| 481 | aagcaccggc taactacgtg ccagcagccg cggtaatacg tagggtgcga gcgttatccg |
| 541 | gatttattgg gcgtaaaggg ctcgtaggcg gttcgtcgcg tccggtgtga aagtccatcg |
| 601 | cctaacggtg gatctgcgcc gggtacgggc gggctggagt gcggtagggg agactggaat |
| 661 | tcccggtgta acggtggaat gtgtagatat cgggaagaac accaatggcg aaggcaggtc |
| 721 | tctgggccgt cactgacgct gaggagcgaa agcgtgggga gcgaacagga ttagataccc |
| 781 | tggtagtcca cgccgtaaac ggtggatgct ggatgtgggg ccctttccac gggtcccgtg |
| 841 | tcggagccaa cgcgttaagc atcccgcctg gggagtacgg ccgcaaggct aaaactcaaa |
| 901 | gaaattgacg ggggcccgca caagcggcgg agcatgcgga ttaattcgat gcaacgcgaa |
| 961 | gaaccttacc tgggcttgac atgtgccgga tcgccgtgga gacacggttt cccttcgggg |
| 1021 | ccggttcaca ggtggtgcat ggtcgtcgtc agctcgtgtc gtgagatgtt gggttaagtc |
| 1081 | ccgcaacgag cgcaaccctc gccgcatgtt gccagcgggt gatgccggga actcatgtgg |
| 1141 | gaccgccggg gtcaactcgg aggaaggtgg ggatgacgtc agatcatcat gccccttacg |
| 1201 | tccagggctt cacgcatgct acaatggccg gtacaacgcg gtgcgacacg gtgacgtggg |
| 1261 | gcggatcgct gaaaaccggt ctcagttcgg atcgcagtct gcaactcgac tgcgtgaagg |
| 1321 | cggagtcgct agtaatcgcg gatcagcaac gccgcggtga atgcgttccc gggccttgta |
| 1381 | cacaccgccc gtcaagtcat gaaagtgggt agcacccgaa gccggtggcc cgacccttgt |
| 1441 | ggggggagcc gtctaaggtg agactcgtga ttgggactaa gtcgtaacaa ggtagccgta |
| 1501 | ccggaaggtg cggctggatc acctcctta |

Bifidobacterium infantis 16S rRNA gene (NCBI/Genbank accession code D86184, SEQ ID
NO: 4)

| | |
|---|---|
| 1 | tttgatcatg gctcaggatg aacgctggcg gcgtgcttaa cacatgcaag tcgaacggga |
| 61 | tccatcgggc tttgcttggt ggtgagagtg gcgaacgggt gagtaatgcg tgaccgacct |
| 121 | gccccataca ccggaatagc tcctggaaac gggtggtaat gccggatgtt ccagttgatc |
| 181 | gcatggtctt ctgggaaagc tttcgcggta tgggatgggg tcgcgtccta tcagcttgac |
| 241 | ggcggggtaa cggcccaccg tggcttcgac gggtagccgg cctgagaggg cgaccggcca |
| 301 | cattgggact gagatacggc ccagactcct acgggaggca gcagtgggga atattgcaca |
| 361 | atgggcgcaa gcctgatgca gcgacgccgc gtgagggatg gaggccttcg ggttgtaaac |
| 421 | ctcttttatc ggggagcaag cgtgagtgag tttacccgtt gaataagcac cggctaacta |
| 481 | cgtgccagca gccgcggtaa tacgtagggt gcaagcgtta tccggaatta ttgggcgtaa |
| 541 | agggctcgta ggcggttcgt cgcgtccggt gtgaaagtcc atcgcttaac ggtggatccg |
| 601 | cgccgggtac gggcgggctt gagtgcggta ggggagactg gaattcccgg tgtaacggtg |
| 661 | gaatgtgtag atatcgggaa gaacaccaat ggcgaaggca ggtctctggg ccgttactga |
| 721 | cgctgaggag cgaaagcgtg gggagcgaac aggattagat accctggtag tccacgccgt |
| 781 | aaacggtgga tgctggatgt ggggcccgtt ccacgggttc cgtgtcggag ctaacgcgtt |
| 841 | aagcatcccg cctggggagt acggccgcaa ggctaaaact caaagaaatt gacgggggcc |
| 901 | cgcacaagcg gcggagcatg cggattaatt cgatgcaacg cgaagaacct tacctgggct |
| 961 | tgacatgttc ccgacgatcc cagagatggg gtttcccttc ggggcgggtt cacaggtggt |
| 1021 | gcatggtcgt cgtcagctcg tgtcgtgaga tgttgggtta agtcccgcaa cgagcgcaac |
| 1081 | cctcgccccg tgttgccagc ggattgtgcc gggaactcac gggggaccgc cggggttaac |
| 1141 | tcggaggaag gtggggatga cgtcagatca tcatgcccct tacgtccagg gcttcacgca |
| 1201 | tgctacaatg gccggtacaa cgggatgcga cgcggcgacg cggagcggat ccctgaaaac |
| 1261 | cggtctcagt tcggatcgca gtctgcaact cgactgcgtg aaggcggagt cgctagtaat |
| 1321 | cgcgaatcag caacgtcgcg gtgaatgcgt tcccgggcct tgtacacacc gcccgtcaag |
| 1381 | tcatgaaagt gggcagcacc cgaagccggt ggcctaaccc cttgtgggat ggagccgtct |
| 1441 | aaggtgaggc tcgtgattgg gactaagtcg taacaaggta gccgtaccgg aaggtgcggc |
| 1501 | tggatcacct cctta |

Bifidobacterium longum 16S rRNA gene (NCBI/Genbank accession code M58739, SEQ ID
NO: 5)*

| | |
|---|---|
| 1 | ttttgtggag ggttcgattc tggctcagga tgaacgctgg cggcgtgctt aacacatgca |
| 61 | agtcgaacgg gatccatcaa gcttgcttgg tggtgagagt ggcgaacggg tgagtaatgc |
| 121 | gtgaccgacc tgccccatac accggaatag ctcctggaaa cgggtggtaa tgccggatgt |
| 181 | tccagttgat cgcatggtct tctggngaaa gcntttcgcg gtatgggatg gggtcgcgtc |
| 241 | ctatcagctt gacggngggg taacggcnna ccgtggcttc gacgggtagc cggcctgaga |
| 301 | gggcgaccgg ccacattggg actgagatac ggcccngact cctacgggag gcagcagtgg |
| 361 | ggaatattgc acaatgggcg caagcctgat gcagcgacgc cgcgtgaggg atggaggcct |
| 421 | tcgggttgta aacctctttt atcggggagc aagcgagagt gagtttaccc gttgaataag |
| 481 | caccggctaa ctacgtgcca gcagccgcgg taatacgtag ggtgcnagcg ttatccggaa |
| 541 | ttattgggcg taaagggctc gtaggcggtt cgtcgcgtcc ggtgtgaaag tccatcgctt |
| 601 | aacggtggat ccgcgccggg tacgggcggg cttgagtgcg gtaggggaga ctggaattcc |
| 661 | cggtgtaacg gtggaatgtg tagatatcgg gaagaacacc aatggcgaag gcaggtctct |
| 721 | gggccgttac tgacgctgag gagcgaaagc gtggggagcg aacaggatta gataccctgg |
| 781 | tagtccacgc cgtaaacggt ggatgctgga tgtggggccn gttccacggg ttccgtgtcg |
| 841 | gagctaacgc gttaagcatc ccgcctgggg agtacggccg caaggctaaa actcaaagaa |
| 901 | attgacgggg gccngcacaa gcggcggagc atgcggatta attcgatgna acgcgaagaa |
| 961 | ccttacctgg gcttgacatg ttcccgacgg tcgtagagat acggcntccc ttcggggcgg |
| 1021 | gttcacaggt ggngcatggt cgtcgtcagc tcgtgtcgtg agatgttggg ttaagtcccg |
| 1081 | caacgagcgc aaccctcgcc ccgtgttgcc agcggattat gccggnaact cacgggnnac |
| 1141 | cgccggggtt aactcggagg aaggtgggga tgacgtcaga tcatcatgcc ccttacgtcc |
| 1201 | agggcttcac gcatgctaca atggccggta caacgggatg cgacgcggcg acgcggagcg |
| 1261 | gatccctgaa aaccngtctc agttcggatc gcagtctgca actcgactgc gtgaaggcgg |
| 1321 | agtcgctagt aatcgcgaat cagcaacgtc gcggtgaatg cgttcccngg ccttgtacac |
| 1381 | accgcccgtc aagncatgaa agtgggcagc acccgaagcc ggtggcctaa ccccttgtgg |
| 1441 | ganggagccg tctaaggtga ggctcgtgat tgggac |

Bifidobacterium breve 16S rRNA gene (NCBI/Genbank accession code AB006658, SEQ ID
NO: 6)

| | |
|---|---|
| 1 | ttcgattctg gctcaggatg aacgctggcg gcgtgcttaa cacatgcaag tcgaacggga |
| 61 | tccatcgggc tttgcttggt ggtgagagtg gcgaacgggt gagtaatgcg tgaccgacct |
| 121 | gccccatgca ccggaatagc tcctggaaac gggtggtaat gccggatgct ccatcacacc |
| 181 | gcatggtgtg ttgggaaagc ctttgcggca tgggatgggg tcgcgtccta tcagcttgat |
| 241 | ggcggggtaa cggcccacca tggcttcgac gggtagccgg cctgagaggg cgaccggcca |
| 301 | cattgggact gagatacggc ccagactcct acgggaggca gcagtgggga atattgcaca |
| 361 | atgggcgcaa gcctgatgca gcgacgccgc gtgagggatg gaggccttcg ggttgtaaac |
| 421 | ctcttttgtt agggagcaag gcactttgtg ttgagtgtac ctttcgaata agcaccggct |
| 481 | aactacgtgc cagcagccgc ggtaatacgt agggtgcaag cgttatccgg aattattggg |
| 541 | cgtaaagggc tcgtaggcgg ttcgtcgcgt ccggtgtgaa agtccatcgc ttaacggtgg |
| 601 | atccgcgccg ggtacgggcg ggcttgagtg cggtagggga gactggaatt cccggtgtaa |
| 661 | cggtggaatg tgtagatatc gggaagaaca ccaatggcga aggcaggtct ctgggccgtt |
| 721 | actgacgctg aggagcgaaa gcgtggggag cgaacaggat tagataccct ggtagtccac |
| 781 | gccgtaaacg gtggatgctg gatgtggggc ccgttccacg ggttccgtgt cggagctaac |
| 841 | gcgttaagca tcccgcctgg ggagtacggc cgcaaggcta aaactcaaag aaattgacgg |
| 901 | gggcccgcac aagcggcgga gcatgcggat taattcgatg caacgcgaag aaccttacct |
| 961 | gggcttgaca tgttcccgac gatcccagag atggggtttc ccttcggggc gggttcacag |
| 1021 | gtggtgcatg gtcgtcgtca gctcgtgtcg tgagatgttg ggttaagtcc cgcaacgagc |
| 1081 | gcaaccctcg ccccgtgttg ccagcggatt gtgccgggaa ctcacggggg accgccgggg |
| 1141 | ttaactcgga ggaaggtggg gatgacgtca gatcatcatg ccccttacgt ccagggcttc |
| 1201 | acgcatgcta caatggccgg tacaacggga tgcgacagtg cgagctggag cggatccctg |
| 1261 | aaaaccggtc tcagttcgga tcgcagtctg caactcgact gcgtgaaggc ggagtcgcta |
| 1321 | gtaatcgcga atcagcaacg tcgcggtgaa tgcgttcccg ggccttgtac acaccgcccg |
| 1381 | tcaagtcatg aaagtgggca gcacccgaag ccggtggcct aaccccttgc gggagggagc |
| 1441 | cgtctaaggt gaggctcgtg attgggacta agtcgtaaca aggtagccgt accggaaggt |
| 1501 | gcggctggat cacctcctta |

Bifidobacterium thermophilum 16S rRNA gene (NCBI/Genbank accession code AB016246,
SEQ ID NO: 7)

| | |
|---|---|
| 1 | agagtttgat catggctcag gatgaacgct ggcggcgtgc ttaacacatg caagtcgaac |
| 61 | gggatcctgc gggctttgcc tgcgggtgag agtggcgaac gggtgagtaa tgcgtgacca |
| 121 | acctgcccca tgctccggaa tagctcctgg aaacgggtgg taatgccgga tgttcccgcg |
| 181 | ccccgcatgg ggtgcgggga aaagcttttg cggcgtggga tggggtcgcg tcctatcagc |
| 241 | ttgttggcgg ggtgagggcc caccaaggct tcgacgggta gccggcctga gaaggcgacc |
| 301 | ggccacattg ggactgagat acggcccaga ctcctacggg aggcagcagt ggggaatatt |
| 361 | gcacaatggg cgcaagcctg atgcagcgac gccgcgtgcg ggatggaggc cttcgggttg |
| 421 | taaaccgctt ttgtttggga gcaagccctt cggggtgagt gtacctttcg aataagcacc |
| 481 | ggctaaatac gtgccagcag ccgcggtaat aagtagggtg cgagcgttat ccggatttat |
| 541 | tgggcgtaaa gggcttgtag gcggtttgtc gcgtccggtg tgaaagtcca tcgcctaacg |
| 601 | gtggatttgc gccgggtacg ggcgggctgg agtgcggtag gggagactgg aattcccggt |
| 661 | gtaacggtgg aatgtgtaga tatcgggaag aacaccaatg gcgaaggcag gtctttgggc |
| 721 | cgttactgac gctgaggagc gaaagcgtgg ggagcgaaca ggattagata ccctggtagt |
| 781 | ccacgccgta aacggtggat gctggatgtg gggcccttcc acgggtcccg tgtcggggcc |
| 841 | aacgcgttaa gcatcccgcc tggggagtac ggccgcaagg ctaaaactca aagaaattga |
| 901 | cgggggcccg cacaagcggc ggagcatgcg gattaattcg atgcaacgcg aaaaacctta |
| 961 | cctgggcttg acatgttccc gacgacggca gagatgtcgt ttcccttcgg ggcgggttca |
| 1021 | caggtggtgc atggtcgtcg tcagctcgtg tcgtgagatg ttgggtcaag tcccgcaacg |
| 1081 | agcgcaaccc tcgccccgtg ttgccagcgc gtcttggcgg gaactcaccg gggaccgccg |
| 1141 | gggtttaccc ggaggaaggt ggggatgacg tcagatcatc atgcccctta cgtccagggc |
| 1201 | ttcacggcat gctacaatgg ccgggtacag gcggggatgc agacatggtg acatggagcg |
| 1261 | ggatccctga aaaccggtct cagttcggga tcggagcgtg caacccggct cggtgaaggc |
| 1321 | ggagtcggct aagtaatcgc ggatcagcaa cgccgcggtg aatgcgttcc cgggccttgt |
| 1381 | acacaccgcc cgtcaagtca tgaaagtggg cagcacccga agccggtggc ctgaccagta |
| 1441 | ttgctggggg gagccgtcta aggtgaggct cgcgattggg agtaagtcgt aacaaggtag |
| 1501 | ccgtaccgga aggtgcggct ggatcacctc ctt |

Bifdobacterium bifidum 16S rRNA gene (NCBI/Genbank accession code M38018, SEQ ID
NO: 8)*

| | |
|---|---|
| 1 | tttttgtgga gggttcgatt ctggctcagg atgaacgctg gcggcgtgct taacacatgc |
| 61 | aagtcgaacg ggatccatca agcttgcttg gtggtgagag tggcgaacgg gtgagtaatg |
| 121 | cgtgaccgac ctgccccatg ctccggaata gctcctggaa acgggtggta atgccgnatg |
| 181 | ttccacatga tcgcatgtga ttgtgggaaa gattctatcg gcgtgggatg gggtcgngtc |
| 241 | ctatcagctt gttggtgagg taacggctca ccaaggcttc gacgggtagc cggcctgaga |
| 301 | gggcgaccgg ccacattggg actgagatac ggcccagact cctacgggag gcagcagtgg |
| 361 | ggaatattgc acaatgggcg caagcctgat gcagcgacgc cgcgtgaggg atggaggcct |
| 421 | tcgggttgta aacctctttt gtttgggagc aagccttcgg gtgagtgtac ctttcgaata |
| 481 | agcgccggct aactacgtgc cagcagccgc ggtaatacgt agggnnnnag cgttatccgg |
| 541 | atttattggg cgtaaagggc tcgtaggcgg ctcgtcgcgt ccggtgtgaa agtccatcgc |
| 601 | ttaacggtgg atctgcgccg ggtacgggcg ggctggagtg cggtagggga gactggaatt |
| 661 | cccggtgtaa cggtggaatg tgtagatatc gggaagaaca ccgatggcga aggcaggtct |
| 721 | ctgggcngtc actgacgctg aggagcnaaa gcgtggggag cgaacaggat tagataccct |
| 781 | ggtagtccac gccgtaaacg gtggacgctg gatgtggggc acgttccacg tgttccgtgt |
| 841 | cggagctaac gcgttaagcg tcccgcctgg ggagtacggc cgcaaggcta aaactcaaag |
| 901 | aaattgacgg gggccngcac aagcggcgga gcatgcggat taattcgaac naacgcgaag |
| 961 | aaccttacct gggcttgaca tgttcccgac gacgccagag atggcgtttc ccttcggggc |
| 1021 | gggttcacag gtggtgcatg gtcgtcgtca gctcgtgtcg tgagatgttg ggttaagtcc |
| 1081 | cgcaacgagc gcaaccctcg ccccgtgttg ccagcacgtt atggtgggaa ctcacgggnn |
| 1141 | accgccgggg ttaacncgga ggaaggtggg gatgacgtca gatcatcatg ccccttacgt |
| 1201 | ccagggcttc acgcatgcta caatggccgg tacagcggga tgcgacatgg cgacatggag |
| 1261 | cggatccctg aaaaccggtc tcagttcgga tcggagcctg caacccggct ccgtgaaggc |
| 1321 | ggagtcgcta gtaatcgcgg atcagcaacg ccgcggtgaa tgcgttcccg ggccttgtac |
| 1381 | acaccgcccg tcaagtcatg aaagtgggca gcacccgaag ccggtggcct aaccccttgt |
| 1441 | gggatggagc cgtctaaggt gaggctcgtg nttgggacta agnngtaaca agnnnnnngt |
| 1501 | accggaagnn nnnnnnngat cacctccttt ct |

Bifidobacterium adolescentis 16S rRNA gene (NCBI/Genbank accession code M58729, SEQ
ID NO: 9)*

| | |
|---|---|
| 1 | nnnnttgtgg agggttcgat tctggctcag gatnaacgct ngcggcgtgc ttaacacatg |
| 61 | caagtcgaac gggatcggct ngagcttgct ccggctgtga gagtggcgaa cgggtgagta |
| 121 | atgcgtgacc gacctgcccc atacaccgga atagctcctg gaaacgggtg gtaatgccgg |
| 181 | atgctccagt tggatgcatg tccttctggg aaagattcta tcggtatggg atggggtcgc |
| 241 | gtcctatcag cttgatggcg gggtaacggc ccnccatggc ttcgacgggn agccggcctg |
| 301 | agagggcgac cggccacatt gggactgaga tacggcccng actcctacgg gaggcagcag |
| 361 | tgggnaatat tgcacaatgg gcgcaagcct aatgcagcga cgccgcgtgc gggatgacgg |
| 421 | ccttcgggtt gtaaaccgct tttgactggg agcaagcctt cggggtgagt gtacctttcg |
| 481 | aataagcacc ggctaactac gtgccagcag ccncggtaat acgtagggtg cnagcgttat |
| 541 | ccggaattat tgggcgtaaa gggctcgtag gcggttcgtc gcgtccggtg tgaaagtcca |
| 601 | tcgcttaacg gtggntccgc gccgggtacg ggcggncttg agtgcggtag ggnagactgg |
| 661 | aattccnggt gtaacggtgg aatgtgtaga tatcgggaag aacaccaatg gcgaaggcag |
| 721 | gtctctgggc ngtnactgac gctgaggagc gaaagcgtgg ggagcgaaca ggattagata |
| 781 | ccctggtagt ccacgccgta aacggtggat gctggatgtg gggaccattc cacggtctcc |
| 841 | gtgtcggagc caacgcgtta agcatcccgc ctggggagta cggccgcaag gctaaaactc |
| 901 | aaagaaattg acgggnnccn ncacaagcgg cngagcatgc ggattaattc gatnnaacgc |
| 961 | gaagaacctt acctgggctt gacatgttcc cgacaggccc cagagatggg nnntccttcg |
| 1021 | ggncgggntc acaggtggng catggtcgtc gtcagctcgt gtcgtgagat gttgggttaa |
| 1081 | gtcccgcaac gagcgcaacc ctcgccctgt gttgccagca cgtcgtggtg gnaactcacg |
| 1141 | ggngaccgcc ggggtcaact cggaggaagg tgggnatgac gtcagatcat catgcccctt |
| 1201 | acgtccaggg cttcacgcat gctacaatgg ccggtacaac gggatgcgac ctcgtgaggg |
| 1261 | ggagcggatc ccttaaaacc ggnctcagtt cggattggag tctgcaaccc gactccatga |
| 1321 | aggcggagtc gctagtaatc gcggatcagc aacgccgcgg tnaatgcgtt cccgggcctt |
| 1381 | gtacacaccg cccgtcaagc catgaaagtg ggtagcaccc gaagccggtg gcccnacctt |
| 1441 | tttgggggga gccgtctaag gtgagnctcg tgatngg |

Bifodbacterium catenulatum 16S rRNA gene (NCBI/Genbank accession code M58732, SEQ
ID NO: 10)*

| | |
|---|---|
| 1 | nnnttttgtg agnggttcga ttctggctca ggatgaacgc tggcggcgtg cttaacacat |
| 61 | gcaagtcgaa cgggatcagg cagcttgctg cctggngaga gtggcgaacg ggnnagtaat |
| 121 | gcgtgaccna cctgccnnat acaccggaat agctcctgga aacgggtggt aatgccggat |
| 181 | gctccgactc ctcgcatggg gtgtcggnaa agatttcatc ggtatgggat ggggtcgngt |
| 241 | cctatcaggt agtcggcggg gtaacggcnn nccgagcctn cgacgggtag ccggcctgag |
| 301 | agggcgaccg gccacattgg gactgagata cggccnngac tcctacggga ggcagcagtg |
| 361 | ggncatattg cacaatgggc gcaagcctna tgcagcgacg cnnngtgcgg gntgacggcc |
| 421 | tncgggttgt aaaccncntt tgatcgggag caagccttcg ggtgagtgta ccnttcgaat |
| 481 | aagcaccggc taactacgtg ccagcagccg cggtaatacg tagggtgcna gcgttatccg |
| 541 | gaattattgg gcgtaaaggg ctcgtaggcg gttcgtcgcg tccggtgtga aagtccatcg |
| 601 | cttaacggtg gatctgcgcc gggtacgggc gggctggagt gcggtagggg ngactggaat |
| 661 | tcccggtgta acggtggaat gtgtagatat cgggaagaac accaatggcg aaggcnggtc |
| 721 | tctgggcngn nactgacgct gaggagcgaa agcgtgggga gcgaacagga ttagataccc |
| 781 | tggtagtcca cgccgtaaac ggtggatgct ggatgtgggg cnngttccac gggttccgtg |
| 841 | tcggagctaa cgcgttaagc atccngcctg gggngtncgg cngcaaggcn nnnncncaaa |
| 901 | gaaattgang ggggccngca caagcggngg agcatgcgga ttnattcgan nnaacgcgaa |
| 961 | gaaccttacc tgggcttgac atgttcccga cagccgtaga gatacggnct cccttcgggg |
| 1021 | cgggnncaca ggtggngcat ggtcgtcgtc ngctcgtgtc gtgagatgtt gggttaagtc |
| 1081 | ccncaacgag cgcaaccctc gccctgtgtt gccgacacgt catgtnggna ctcacgggnn |
| 1141 | accgccgggg tcaactcgga ggaaggtggg gatgacgtca gatcatcatg ccccttacgt |
| 1201 | ccagggcttc acgcatgcta caatggccgg tacaacggga tgcgacatgg cgacatggag |
| 1261 | cggatccctg aaaaccggnc tcagttcgga ttggagtctg caacccgact ccatgaaggc |
| 1321 | ggagtcgcta gtaatcgcgg atcagcaacg ccgcggtgaa tgcgttcccg ggccttgtac |
| 1381 | acaccgcncg tcaagncatg aaagtgggta gcacccgaag ccggtggcct nacccnttgt |
| 1441 | gggatggagc cgtctaaggt gagactcgtg attgggac |

Bifdobacterium pseudocatenulatum 16S rRNA gene (NCBI/Genbank accession code D86187,
SEQ ID NO: 11)

| | |
|---|---|
| 1 | gtttcgattc tggctcagga tgaacgctgg cggcgtgctt aacacatgca agtcgaacgg |
| 61 | gatccatcag gctttgcttg gtggtgagag tggcgaacgg gtgagtaatg cgtgaccgac |
| 121 | ctgccccata caccggaata gctcctggaa acgggtggta atgccggatg ctccgactcc |
| 181 | tcgcatgggg tgtcgggaaa gatttcatcg gtatgggatg gggtcgcgtc ctatcaggta |
| 241 | gtcggcgggg taacggccca ccgagcctac gacgggtagc cggcctgaga gggcgaccgg |
| 301 | ccacattggg actgagatac ggcccagact cctacgggag gcagcagtgg ggaatattgc |
| 361 | acaatgggcg caagcctgat gcagcgacgc cgcgtgcggg atgacggcct tcgggttgta |
| 421 | aaccgctttt gatcgggagc aagccttcgg gtgagtgtac ctttcgaata agcaccggct |
| 481 | aactacgtgc cagcagccgc ggtaatacgt agggtgcaag cgttatccgg aattattggg |
| 541 | cgtaaagggc tcgtaggcgg ttcgtcgcgt ccggtgtgaa agtccatcgc ttaacggtgg |
| 601 | atctgcgccg ggtacgggcg ggctggagtg cggtagggga gactggaatt cccggtgtaa |
| 661 | cggtggaatg tgtagatatc gggaagaaca ccaatggcga aggcaggtct ctgggccgtt |
| 721 | actgacgctg aggagcgaaa gcgtggggag cgaacaggat tagataccct ggtagtccac |
| 781 | gccgtaaacg gtggatgctg gatgtggggc ccgttccacg ggttccgtgt cggagctaac |
| 841 | gcgttaagca tcccgcctgg ggagtacggc cgcaaggcta aaactcaaag aaattgacgg |
| 901 | gggcccgcac aagcggcgga gcatgcggat taattcgatg caacgcgaag aaccttacct |
| 961 | gggcttgaca tgttcccgac agccgtagag atatggcctc ccttcggggc gggttcacag |
| 1021 | gtggtgcatg gtcgtcgtca gctcgtgtcg tgagatgttg ggttaagtcc cgcaacgagc |
| 1081 | gcaaccctcg ccctgtgttg ccagcacgtc atggtgggaa ctcacggggg accgccgggg |
| 1141 | tcaactcgga ggaaggtggg gatgacgtca gatcatcatg ccccttacgt ccagggcttc |
| 1201 | acgcatgcta caatggccgg tacaacggga tgcgacacgg cgacgtggag cggatccctg |
| 1261 | aaaaccggtc tcagttcgga ttggagtctg caacccgact ccatgaaggc ggagtcgcta |
| 1321 | gtaatcgcgg atcagcaacg ccgcggtgaa tgcgttcccg ggccttgtac acaccgcccg |
| 1381 | tcaagtcatg aaagtgggta gcacccgaag ccggtggcct aaccctttgt ggatggagcc |
| 1441 | gtctaaggtg agactcgtga ttgggactaa gtcgtaacaa ggtagccgta ccggaaggtg |
| 1501 | cggctggatc acctcctta |

*“n” refers to a, t, c, or g.

In another particularly preferred embodiment, the Anaerobutyricum soehngenii or relative thereof and/or the at least one Bifidobacterium species according to the disclosure, is combined with at least one Akkermansia species, preferably wherein the at least one Akkermansia species is pasteurized or has been subjected to pasteurization (i.e., heating to 55-99, preferably 65-80 degrees Celsius for 5-60 seconds or 1-60 minutes, preferably 60-80 degrees Celsius for 20-40minutes, more preferably 65-75 degrees Celsius for 25-35 minutes). It was found that this is a further synergistic combination, leading to an unexpected reduction in hepatic necroinflammatory activity score.

The at least one Akkermansia species may be administered separately, sequentially or simultaneously with Anaerobutyricum soehngenii or relative thereof and/or at least one Bifidobacterium species. Accordingly, the Akkermansia species may be comprised in the same or in a separate composition with respect to Anaerobutyricum soehngenii or relative thereof and/or the at least one Bifidobacterium species.

Preferably, the at least one Akkermansia species according to the present disclosure is Akkermansia muciniphila or relative thereof having a 16S rRNA sequence with at least 90, 95, 97, 99, or 100% sequence identity with SEQ ID NO: 12.

Akkermansia is a genus in the phylum Verrucomicrobia. It was found that Akkermansia species improve intestinal mucosal barrier function, or intestinal barrier function, which refers to the property of the intestinal mucosa that ensures adequate containment of undesirable luminal contents within the intestine while preserving the ability to absorb nutrients. Its role in protecting the mucosal tissues and circulatory system from exposure to pro-inflammatory molecules, such as microorganisms, toxins, and antigens is vital for the maintenance of health and well-being. Accordingly, Akkermansia species may prevent or be used for treating intestinal mucosal barrier dysfunction, which has been implicated in numerous health conditions such as: food allergy, microbial infection, irritable bowel syndrome, inflammatory bowel disease, celiac disease, metabolic syndrome, non-alcoholic fatty liver disease, diabetes, and septic shock. See Collado et al., 2007 (Appl. Environ. Microbiol. 2007 December; 73 (23): 7767-70). Or see Appl. Environ. Microbiol. 2020 Mar. 18; 86 (7): e03004-19.

The at least one Akkermansia species of the present disclosure preferably includes one or more of:
Akkermansia muciniphila (able to assimilate HMO) or relative thereof having a 16S rRNA gene with at least 90, 95, 97, 98, 99, 100% sequence identity with the 16S rRNA gene sequence of the type strain of Akkermansia muciniphila (NCBI accession code AY271254, SEQ ID NO:12).
Akkermansia glycanipila or relative thereof having a 16S IRNA gene with at least 90, 95, 97, 98, 99, 100% sequence identity with the 16S rRNA gene sequence of the type strain of Akkermansia glycanipila (NCBI accession code NR152695, SEQ ID NO:13).

Akkermansia muciniphila 16S rRNA gene (NCBI/Genbank accession code AY271254, SEQ ID
NO: 12)

| | |
|---|---|
| 1 | aacgaacgct ggcggcgtgg ataagacatg caagtcgaac gagagaattg ctagcttgct |
| 61 | aataattctc tagtggcgca cgggtgagta acacgtgagt aacctgcccc cgagagcggg |
| 121 | atagccctgg gaaactggga ttaataccgc atagtatcga aagattaaag cagcaatgcg |
| 181 | cttggggatg ggctcgcggc ctattagtta gttggtgagg taacggctca ccaaggcgat |
| 241 | gacgggtagc cggtctgaga ggatgtccgg ccacactgga actgagacac ggtccagaca |
| 301 | cctacgggtg gcagcagtcg agaatcattc acaatggggg aaaccctgat ggtgcgacgc |
| 361 | cgcgtggggg aatgaaggtc ttcggattgt aaacccctgt catgtgggag caaattaaaa |
| 421 | agatagtacc acaagaggaa gagacggcta actctgtgcc agcagccgcg gtaatacaga |
| 481 | ggtctcaagc gttgttcgga atcactgggc gtaaagcgtg cgtaggctgt ttcgtaagtc |
| 541 | gtgtgtgaaa ggcgcgggct caacccgcgg acggcacatg atactgcgag actagagtaa |
| 601 | tggaggggga accggaattc tcggtgtagc agtgaaatgc gtagatatcg agaggaacac |
| 661 | tcgtggcgaa ggcgggttcc tggacattaa ctgacgctga ggcacgaagg ccaggggagc |
| 721 | gaaagggatt agatacccct gtagtcctgg cagtaaacgg tgcacgcttg gtgtgcgggg |
| 781 | aatcgacccc ctgcgtgccg gagtaacgcg ttaagcgtgc cgcctgggga gtacggtcgc |
| 841 | aagattaaaa ctcaaagaaa ttgacgggga cccgcacaag cggtggagta tgtggcttaa |
| 901 | ttcgatgcaa cgcgaagaac cttacctggg cttgacatgt aatgaacaac atgtgaaagc |
| 961 | atgcgactct tcggaggcgt tacacaggtg ctgcatggcc gtcgtcagct cgtgtcgtga |
| 1021 | gatgtttggt taagtccagc aacgagcgca acccctgttg ccagttacca gcacgtgaag |
| 1081 | gtggggactc tggcgagact gcccagatca actgggagga aggtggggac gacgtcaggt |
| 1141 | cagtatggcc cttatgccca gggctgcaca cgtactacaa tgcccagtac agagggggcc |
| 1201 | gaagccgcga ggcggaggaa atcctaaaaa ctgggcccag ttcggactgt aggctgcaac |
| 1261 | ccgcctacac gaagccggaa tcgctagtaa tggcgcatca gctacggcgc cgtgaatacg |
| 1321 | ttcccgggtc ttgtacacac cgcccgtcac atcatggaag ctggtcgcac ccgaagtatc |
| 1381 | tgaagccaac cgcaaggagg cagggtccta aggtgagact ggtaactggg atg |

Akkermansia glycanipila 16S rRNA gene (NCBI/Genbank accession code NR152695, SEQ ID
NO: 13)

| | |
|---|---|
| 1 | aacgaacgct ggcggcgtgg ataagacatg caagtcgaac ggagaagcaa tagcttgcta |
| 61 | atgcttctta gtggcgcacg ggtgagtaac acgtgagcaa cctgccttcg agacgggaat |
| 121 | agccctggga aaccgggatt aatgcccgat agactcgcaa gagtaaacgc agcaatgcgc |
| 181 | ttgaagaggg gctcgcggcc tattagttag ttggtgaggt aacggctcac caaggcgatg |
| 241 | acgggtagcc ggtctgagag gatgtccggc cacactggaa ctgagacacg gtccagacac |
| 301 | ctacgggtgg cagcagtcga gaatcattca caatggggga aaccctgatg gtgcgacgcc |
| 361 | gcgtggggga agaaggtctt cggattgtaa acccctgtca tgtgggagca aggcgcaagc |
| 421 | ttgatagtac cacaagagga agagacggct aactctgtgc cagcagccgc ggtaatacag |
| 481 | aggtctcaag cgttgttcgg aatcactggg cgtaaagggt acgtaggctg catcataagt |
| 541 | cgggcgtgaa aggcaggggc tcaacccctg gagtgcgctt gatactgtga tgctagagtc |
| 601 | atggaggggg aaccggaact ctcggtgtag cagtgaaatg cgtagatatc gagaagaaca |
| 661 | ctcgtggcga aggcgggttc ctggacatgt actgacgctg aggtacgaag gctaggggag |
| 721 | cgaaagggat tagatacccc tgtagtccta gcagtaaacg gtgcacgctt ggtgtgtggg |
| 781 | gaatcgaccc cccacgtgcc ggagcaaacg cgttaagcgt gccgcctggg gagtacggtc |
| 841 | gcaagattaa aactcaaaga aattgacggg gacccgcaca agcggtggag tatgtggctt |
| 901 | aattcgatgc aacgcgaaga accttacctg ggcttgacat gtgatgaaca acatgtgaaa |
| 961 | gcatgtgaca cctcggtggc gtcacacagg tgctgcatgg ccgtcgtcag ctcgtgtcgt |
| 1021 | gagatgtttg gttaagtcca gcaacgagcg caacccctgt tgccagttac cagcacgtta |
| 1081 | tggtggggac tctggcgaga ctgcccagat caactgggag gaaggtgggg acgacgtcag |
| 1141 | gtcagtatgg cccttatgcc cagggctgca cacgtactac aatgcccagt acagagggta |
| 1201 | ccgaacccgc gagggggagg caatccatga aaactgggcc cagttcggat tgtaggctgc |
| 1261 | aactcgccta catgaagatg gaatcgctag taatggcgca tcagctacgg cgccgtgaat |
| 1321 | acgttcccgg gtcttgtaca caccgcccgt cacatcatgg aagccggtcg cacccgaagt |
| 1381 | atctgaagcc aaccgcaagg aggcagggtc ctaaggtgag actggtaact gggatgaa |

In another particularly preferred embodiment, the Anaerobutyricum soehngenii or relative thereof and/or the at least one Bifidobacterium species and/or the at least one Akkermansia species according to the disclosure is combined with at least one Lactobacillus species. It was found that this is a further synergistic combination, leading to an unexpected reduction in hepatic necroinflammatory activity score.

The at least one Lactobacillus species may be administered separately, sequentially or simultaneously with Anaerobutyricum soehngenii or relative thereof and/or at least one Bifidobacterium species and/or at least one Akkermansia species. Accordingly, the Lactobacillus species may be comprised in the same or in a separate composition with respect to Anaerobutyricum soehngenii or relative thereof and/or the at least one Bifidobacterium species and/or the at least one Akkermansia species.

The Lactobacillus species is preferably chosen from:
Lactobacillus acidophilus or relative thereof having a 16S rRNA sequence with at least 90, 95, 97, 99, 100% sequence identity with SEQ ID NO:14;
Lactobacillus casei or relative thereof having a 16S rRNA sequence with at least 90, 95, 97, 99, 100% sequence identity with SEQ ID NO:15;
Lactobacillus reuteri or relative thereof having a 16S IRNA sequence with at least 90, 95, 97, 99, 100% sequence identity with SEQ ID NO: 16; and/or
Lactobacillus rhamnosus or relative thereof having a 16S rRNA sequence with at least 90, 95, 97, 99, 100% sequence identity with SEQ ID NO:17.

Lactobacillus acidophilus 16S rRNA sequence (NCBI NR_043182.1)(SEQ ID NO: 14)

| | |
|---|---|
| 1 | tcctggctca ggacgaacgc tggcggcgtg cctaatacat gcaagtcgag cgagctgaac |
| 61 | caacagattc acttcggtga tgacgttggg aacgcgagcg gcggatgggt gagtaacacg |
| 121 | tggggaacct gccccatagt ctgggatacc acttggaaac aggtgctaat accggataag |
| 181 | aaagcagatc gcatgatcag cttataaaag gcggcgtaag ctgtcgctat gggatggccc |
| 241 | cgcggtgcat tagctagttg gtagggtaac ggcctaccaa ggcaatgatg catagccgag |
| 301 | ttgagagact gatcggccac attgggactg agacacggcc caaactccta cgggaggcag |
| 361 | cagtagggaa tcttccacaa tggacgaaag tctgatggag caacgccgcg tgagtgaaga |
| 421 | aggttttcgg atcgtaaagc tctgttgttg gtgaagaagg atagaggtag taactggcct |
| 481 | ttatttgacg gtaatcaacc agaaagtcac ggctaactac gtgccagcag ccgcggtaat |
| 541 | acgtaggtgg caagcgttgt ccggatttat tgggcgtaaa gcgagcgcag gcggaagaat |
| 601 | aagtctgatg tgaaagccct cggcttaacc gaggaactgc atcggaaact gtttttcttg |
| 661 | agtgcagaag aggagagtgg aactccatgt gtagcggtgg aatgcgtaga tatatggaag |
| 721 | aacaccagtg gcgaaggcgg ctctctggtc tgcaactgac gctgaggctc gaaagcatgg |
| 781 | gtagcgaaca ggattagata ccctggtagt ccatgccgta aacgatgagt gctaagtgtt |
| 841 | gggaggtttc cgcctctcag tgctgcagct aacgcattaa gcactccgcc tggggagtac |
| 901 | gaccgcaagg ttgaaactca aaggaattga cgggggcccg cacaagcggt ggagcatgtg |
| 961 | gtttaattcg aagcaacgcg aagaacctta ccaggtcttg acatctagtg caatccgtag |
| 1021 | agatacggag ttcccttcgg ggacactaag acaggtggtg catggctgtc gtcagctcgt |
| 1081 | gtcgtgagat gttgggttaa gtcccgcaac gagcgcaacc cttgtcatta gttgccagca |
| 1141 | ttaagttggg cactctaatg agactgccgg tgacaaaccg gaggaaggtg gggatgacgt |
| 1201 | caagtcatca tgccccttat gacctgggct acacacgtgc tacaatggac agtacaacga |
| 1261 | ggagcaagcc tgcgaaggca agcgaatctc ttaaagctgt tctcagttcg gactgcagtc |
| 1321 | tgcaactcga ctgcacgaag ctggaatcgc tagtaatcgc ggatcagcac gccgcggtga |
| 1381 | atacgttccc gggccttgta cacaccgccc gtcacaccat gggagtctgc aatgcccaaa |
| 1441 | gccggtggcc taaccttcgg gaaggagccg tctaaggc |

Lactobacillus casei 16S rRNA sequence (NCBI MT994696)(SEQ ID NO: 15)

| | |
|---|---|
| 1 | gttggagaag aatggtcggc agagtaactg ttgtcggcgt gacggtatcc aaccagaaag |
| 61 | ccacggctaa ctacgtgcca gcagccgcgg taatacgtag gtggcaagcg ttatccggat |
| 121 | ttattgggcg taaagcgagc gcaggcggtt ttttaagtct gatgtgaaag ccctcggctt |
| 181 | aaccgaggaa gcgcatcgga aactgggaaa cttgagtgca gaagaggaca gtggaactcc |
| 241 | atgtgtagcg gtgaaatgcg tagatatatg gaagaacacc agtggcgaag gcggctgtct |
| 301 | ggtctgtaac tgacgctgag gctcgaaagc atgggtagcg aacaggatta gataccctgg |
| 361 | tagtccatgc cgtaaacgat gaatgctagg tgttggaggg tttccgccct tcagtgccgc |
| 421 | agctaacgca ttaagcattc cgcctgggga gtacgaccgc aaggttgaaa ctcaaaggaa |
| 481 | ttgacggggg cccgcacaag cggtggagca tgtggtttaa ttcgaagcaa cgcgaagaac |
| 541 | cttaccaggt cttgacatct ttttgatcac tgagagatca ggtttcccct tcgggggcaa |
| 601 | aatgacaggt ggtgcatgtt gtcgtcagct cgtgtcgtga gatgttgggt taagtcccgc |
| 661 | aacgagcgct a |

Lactobacillus reuteri 16S rRNA sequence (NCBI NR_025911)(SEQ ID NO: 16)

| | |
|---|---|
| 1 | agagtttgat cctggctcag gatgaacgcc ggcagtgtgc ctaatacatg caagtcgtac |
| 61 | gcactggccc aactaattga tggtgcttgc tgaattgacg atggatcacc agtgagtggc |
| 121 | ggacgggtga gtaacacgta ggtaacctgc cccggagcgg ggaataacat ttggaaacag |
| 181 | atgctaatac cgcataacaa caaaagccgc atggtttttc tggaaagatg gctttggcta |
| 241 | tcactctggg atggacctgc ggtgcattta gctagttggt aaggtaacgg cttacccaag |
| 301 | gcgatgatgc atagccgagt tgagagactg atcggccaca atgggaactg agacacggtc |
| 361 | cataacttct acgggaggca gcagtaggga atcttccaca atgggcgcaa gctgatggag |
| 421 | caacaccgcg ttattaagaa agggtttcgg ccgcttaaac tctgttgttg gagaagaacg |
| 481 | tgcgttagag taactgttac gcagtgacgg tatccaacca gaaagtcacg gctaactacg |
| 541 | tgccagcagc cgcggtaata cgtaggtggc aagcgttatc cggatttatt gggcgtaaag |
| 601 | cgagcgcagg cggttgctta ggtctgatgt ggaaactcgg cttaaccgaa gaagtgcatc |
| 661 | ggaaaccggg cgacttgagt gcagaagagg acagtggaac tccatgtgta gcggtggaat |
| 721 | gcgtagatat atggaagaac accagtggcg aaggcggctg tctggtctgc aactgacgct |
| 781 | gaggctcgaa agcatgggta gcgaacagga ttagataccc tggtagtcca tgccgtaaac |
| 841 | gatgagtgct aggtgttgga gggtttccgc ccttcagtgc ctgttctaac gcattaatgc |
| 901 | actccgcctg gggagtacga ccgcaaggtt gaaactcaaa ggaattgacg ggggcccgca |
| 961 | caagcggtga agcatgtggt ttaattcgaa gctacgcgaa gaaccttacc aggtcttgac |
| 1021 | atcttgcgct aaccttagag ataaggcgtt cccttcgggg acgttaatga caggtggtgc |
| 1081 | atggtcgtcg tcagctcgtg tcgtgagatg ttgggttaag tcccgcaacg agcgcaaccc |
| 1141 | ttgttactag ttgccagcat taagttgggg actctagtga gactgccggt gacaaaccgg |
| 1201 | aggaaggtgg ggacgacgtc agatcatcat gccccttatg accctgggct acacacgtgc |
| 1261 | tacaatggac ggtacaacga gtcgcaaact cgcgagagta agctaatctc ttaaagccgt |
| 1321 | tctcagttcg gactgtaggc tgcaactcgc ctacacgaag tcggaatcgc tagtaatcgc |
| 1381 | ggatcagcat gccgcggtga atacgttccc gggccttgta cacaccgccc gtcacaccat |
| 1441 | gggagtttgt aacgcccaaa gttcggtggc ctaaccttta tggacgggta ccctaaggcg |
| 1501 | ggacagatga tctggggtga agtcgtaaca aggta |

Lactobacillus rhamnosus 16S rRNA sequence (NCBI NR_043408.1)(SEQ ID NO: 17)

| | |
|---|---|
| 1 | grtsaacgct sgcggcgtgc ctaatacatg caagtcgaac gagttctgat tattgaaagg |
| 61 | tgcttgcatc ttgatttaat tttgaacgag tggcggacgg gtgagtaaca cgtgggtaac |
| 121 | ctgcccttaa gtgggggata acatttggaa acagatgcta ataccgcata aatccaagaa |
| 181 | ccgcatggtt cttggctgaa agatggcgta agctatcgct tttggatgga cccgcggcgt |
| 241 | attagctagt tggtgaggta acggctcacc aaggcaatga tacgtagccg aactgagagg |
| 301 | ttgatcggcc acattgggac tgagacacgg cccaaactct acgggaggca gcagtaggga |
| 361 | atcttccaca atggacgcaa gtctgatgga gcaacgccgc gtgagtnaag aaggctttcg |
| 421 | ggtcgtaaaa ctctgttgtt ggagaagaat ggtcggcaga gtaactgttg tcggcgtgac |
| 481 | ggtatccaac cagaaagcca cggctaacta cgtgccagca gccgcggtaa tacgtaggtg |
| 541 | gcaagcgtta tccggattta ttgggcgtaa agcgagcgca ggcggttttt taagtctgat |
| 601 | gtgaaagccc tcggcttaac cgaggaagtg catcggaaac tgggaaactt gagtncagaa |
| 661 | gaggacagtg gaactccatg tgtagcggtg aaatgcgtag atatatggaa gaacaccagt |
| 721 | ggcgaaggcg gctgtctggt ctgtaactga cgctgaggct cgaaagcatg ggtagcgaac |
| 781 | aggattagat accctggtag tccatgccgt aaacgatgaa tgctaggtgt tggagggttt |
| 841 | ccgcccttca gtgccgcagc taacgcatta agcattccgc ctggggagta cgaccgcaag |
| 901 | gttgaaactc aaaggaattg acgggggccc gcacaagcgg tggagcatgt ggtttaattc |
| 961 | gaagcaacgc gaagaacctt accaggtctt gacatctttt gatcacctga gagatcaggt |
| 1021 | ttccccttcg ggggcaaaat gacaggtggt gcatggttgt cgtcagctcg tgtcgtgaga |
| 1081 | tgttgggtta agtcccgcaa cgagcgcaac ccttatgact agttgccagc atttagttgg |
| 1141 | gcactctagt aagactgccg gtgacaaacc ggaggaaggt ggggatgacg tcaaatcatc |
| 1201 | atgcccctta tgacctgggc tacacacgtg ctacaatgga tggtacaacg agttgcgaga |
| 1261 | ccgcgaggtc aagctaatct cttaaagcca ttctcagttc ggactgtagg ctgcaactcg |
| 1321 | cctacacgaa gtcggaatcg ctagtaatcg cggatcagca cgccgcggtg aatacgttcc |
| 1381 | cgggccttgt acacaccgcc cgtcacacca tgagagtttg taacacccga agccggtggc |
| 1441 | gtaacccttt tagggagcga gccgtctaag gtgggncaaa tgattagggt gaagtcgtaa |
| 1501 | caaggtagcc gtaggagaac c |

In a preferred embodiment, the present disclosure excludes the use (for example, by co-administration) of any Ruminococcus species (for example, Ruminococcus flavefaciens, R. torques or R. faecis) any Faecalibacterium species (for example, Faecalibacterium prausnitzii), and/or any Prevotella species such as Prevotella copri.

The present disclosure may include or exclude any Anaerostipes species (particularly Anaerostipes rhamnisovorans) or any Faecalibacterium species (for example, Faecalibacterium prausnitzii) for improved effect in the prevention and/or treatment according to the present disclosure.

It is envisaged that the Anaerobutyricum soehngenii or relative thereof, Bifidobacterium species, Akkermansia species and/or Lactobacillus species as according to the present disclosure is/are comprised in fecal matter.

The Anaerobutyricum soehngenii or relative thereof, Bifidobacterium species, Akkermansia species and/or Lactobacillus species according to the present disclosure may be or be derived from fecal matter, e.g., obtained from one or more donor subjects. The term “donor” as used herein denotes a subject who donates fecal matter. The fecal matter according to the present disclosure is thus derived from the donor and may be administered to a recipient. Optionally after processing, the fecal matter is administered to the recipient. The one or more donor subjects are preferably mammal, preferably human. Also, the recipient is preferably a mammal, preferably a human.

Preferably the fecal matter is obtained from at least one healthy (human) donor, more preferably at least one (human) donor following (or who has followed) a vegetarian diet, most preferably a vegan diet. A vegetarian diet does not include any meat, poultry or seafood, or at most 0.1, 0.5, 1 kg meat, poultry or seafood per month. A vegan diet does not include any meat, poultry, seafood or any food from animal origin, or at most 0.1, 0.5, 1 kg meat, poultry or seafood or food from animal origin per month. A healthy donor may, for example, be regarded as a donor not having a condition as mentioned in Table 1 of Lise Sofie et al. (2019, Transfusion and Apheresis Science, Volume 58, Issue 1, P113-116).

Selected donor subjects preferably have a BMI between 18-27, preferably between 20 to 25 kg/m2. The term “Body Mass Index” or “BMI” as used herein denotes a value derived from dividing the mass of a person by the square of the person's body height, expressed in kg/m².

Selected donor subjects preferably have an age below 30 years or below 35 years. The at least one donor subject, for example, has an age between 18 and 30 years, such as 20 to 25years. In addition or alternatively, selected donor(s) follow (or have followed) a diet rich in prebiotic fiber (that increases butyrate production in stools), such as WholeFiber, see WO2021/204719 (e.g., at least 0.1, 0.5, 1 kg prebiotic fiber per month).

Additionally or alternatively, the at least one donor subject has a relative abundance of Bifidobacteriales species in the fecal matter of at least 0.1, 0.2, 0.3, 0.4, 0.5, 0.6, 0.7, 0.8, 0.9 or 1, 2, 3, 4, 5, 6, 7, 8, 9, 10, 15, 20, 25, 30% (as compared to the number of species of other genera). Additionally or alternatively, the at least one donor subject has a relative abundance of Akkermansia species in the fecal matter of at least 0.1, 0.2, 0.3, 0.4, 0.5, 0.6, 0.7, 0.8, 0.9 or 1, 2, 3, 4, 5, 6, 7, 8, 9, 10, 15, 20, 25, 30% (as compared to the number of species of other genera).

In a preferred embodiment, at least 10⁸, or 10⁸ cells of the Anaerobutyricum soehngenii or relative thereof are comprised in the fecal matter. Similarly, at least 10⁸, or 10⁸ cells of the Bifidobacterium species are comprised in the fecal matter. Similarly, at least 10⁸, or 10⁸ cells of the Akkermansia species are comprised in the fecal matter. Similarly, at least 10⁸, or 10⁸ cells of the Lactobacillus species are comprised in the fecal matter.

In other words, the Anaerobutyricum soehngenii or relative thereof, Bifidobacterium species, Akkermansia species and/or Lactobacillus species as according to the present disclosure is preferably enriched in the fecal matter, i.e., the number of Anaerobutyricum soehngenii or relative thereof, Bifidobacterium species, Akkermansia species and/or Lactobacillus species cells is higher than in prior art fecal matter, for example, Anaerobutyricum soehngenii or relative thereof, Bifidobacterium species, Akkermansia species and/or Lactobacillus species cells have been added to the fecal matter, or the fecal matter has been exposed to conditions favoring growth of the Anaerobutyricum soehngenii or relative thereof, Bifidobacterium species, Akkermansia species and/or Lactobacillus species. If the Anaerobutyricum soehngenii or relative thereof, Bifidobacterium species, Akkermansia species and/or Lactobacillus species according to the present disclosure is comprised in fecal matter, preferably at least at least 10⁴, 10⁵, 2×10⁵, 3×10⁵, 4×10⁵, 5×10⁵, 6×10⁵, 7×10⁵, 8×10⁵, 9×10⁵, 10⁶, 2×10⁶, 3×10⁶, 4×10⁶, 5×10⁶, 6×10⁶, 7×10⁶, 8×10⁶, 9×10⁶, 10⁷, 2×10⁷, 3×10⁷, 4×10⁷, 5×10⁷, 6×10⁷, 7×10⁷, 8×10⁷, 9×10⁷, 10⁸, 10⁹, 10¹⁰, 10¹¹, 10¹², 10¹³ cells are comprised in the fecal matter, for example, per ml or per g fecal matter. Preferably, the Anaerobutyricum soehngenii or relative thereof, Bifidobacterium species, Akkermansia species and/or Lactobacillus species is/are the first, second, third, fourth, fifth, sixth, seventh, eighth, ninth, and/or tenth most dominant bacterial species in the fecal matter, i.e., has the highest cell count in comparison to other bacterial species contained in the fecal matter, or is at least in the top 10.

Preferably, in case the composition according to the present disclosure is fecal matter, the fecal matter can be feces or part thereof, preferably a purified part thereof. By purifying the fecal matter, the fecal matter can be more conveniently administered. In a particular embodiment, 50-150 mg fecal matter sample may be combined with 5-15 mL isotonic saline containing, e.g., 10% glycerol and can be frozen at −80 C until delivery. For example, 1 mL may be mixed with mother's own milk or pasteurized bank milk to a total volume of 10 mL, and 5 mL can be administered to the recipient.

A part of fecal matter as used herein denotes one or more specific groups of components including, but not limited to: enzymes, proteins, lipids, molecules, microorganisms, viruses, bacteria, fungi, yeast, archaea, compounds, complexes, solids, liquids, particles, and fibers.

A purified part of fecal matter as used herein denotes that undesired groups of components are not present in the fecal matter.

Preferably, the fecal matter for use according to the disclosure is comprised in liquid medium and/or does not comprise solids having a diameter of more than 10, 25, 50, 75, 100, 200, 400, 600, 800, or 1000 μm, preferably obtained by mixing allogenic feces with aqueous medium and subsequent filtering and/or centrifugation. This greatly reduces the viscosity and enhances flow of the fecal matter, facilitating administration of the fecal matter to the receiving subject. The liquid medium can comprise water, or another type of liquid, which may be supplemented with other components, such as salts, to provide an isotonic solution.

According to one aspect of the disclosure, the fecal matter according to the disclosure is comprised in a composition, such as a pharmaceutical composition, more preferably a liquid dosage form, facilitating administration of the fecal matter to a recipient.

It is further envisaged that the fecal matter according to the present disclosure is present in lyophilized and/or microencapsulated form (to protect from gastric environment). The use according to the disclosure may involve 1, 2, 3, 4, 5, 6, 7, 8, 9, 10 separate administrations of fecal matter obtained from the at least one donor subject to the recipient, preferably with intervals of at least 1, 2, 3, 4, 5, 6, 7, 8 weeks between the separate administrations.

Alternatively, the Anaerobutyricum soehngenii or relative thereof, Bifidobacterium species, Akkermansia species and/or Lactobacillus species as according to the present disclosure is/are not comprised in fecal matter.

The at least one Anaerobutyricum soehngenii or relative thereof, the at least one Bifidobacterium species, the at least one Akkermansia species and/or the at least one Lactobacillus species as according to the present disclosure may be comprised in a composition.

The composition according to the present disclosure may be administered by enteral, preferably by oral, nasal or rectal administration, and/or by nasoduodenal tube administration.

The composition according to the present disclosure may be used as medicament and/or accompanied by a physiologically acceptable carrier, which may be any inert carrier. For instance, non-limiting examples of suitable physiologically or pharmaceutically acceptable carriers include any well-known physiological or pharmaceutical carriers, buffers, diluents, and excipients. It will be appreciated that the choice for a suitable physiological carrier will depend upon the intended mode of administration of the composition as taught herein (e.g., oral). The skilled person knows how to select a physiologically acceptable carrier, which is suitable for or compatible with the compositions for use as taught herein.

It is envisaged that the composition according to the present disclosure is comprised in and/or encapsulated by an (enteric) coating, preferably wherein the coating does not dissolute and/or disintegrate in the gastric environment of the recipient. Such coating may help the composition to reach the intended site for delivery, e.g., the duodenum, without suffering breakdown due to the acidic environment of the stomach. Preferred (enteric) coatings work by presenting a surface that is stable at the highly acidic pH found in the stomach but breaking down more rapidly at a lower pH. For example, it will not dissolve in the gastric acids of the stomach (pH ˜3), but it will dissolve in the alkaline (pH 7-9) environment present in the small intestine, or duodenum.

In an embodiment, the present disclosure is concerned with the composition for use as a probiotic. Accordingly, “probiotics,” as used herein, refers to microorganisms such as intestinal bacteria, which, when administered or ingested in effective amounts, confer health benefits to the host (e.g., humans or mammals). Preferably, probiotics should be alive or viable when administered to a subject so as to allow the probiotics to colonize the large intestine of the host. However, under certain conditions, probiotics may also be dead when administered provided that substances produced by the probiotics still exert probiotic, beneficial effects on the host.

In an embodiment, the present combination as taught herein may be for use as a symbiotic. The term “symbiotic” or “symbiotic products,” as used herein, generally refers to compositions and/or nutritional supplements combining probiotics and one or more compounds that promote the growth and/or activity of GI microorganisms, such as prebiotics, into one product. The symbiotic beneficially affects the host by improving the survival and colonization of the probiotic in the GI tract, by selectively stimulating the growth and/or by activating the metabolism of the probiotic, thus improving host welfare. The skilled person is well-acquainted with symbiotics and knows how to select ingredients that may be combined into a symbiotic.

Furthermore, it was found that micro-encapsulation of the at least one Anaerobutyricum soehngenii or relative thereof, the at least one Bifidobacterium species, the at least one Akkermansia species and/or the at least one Lactobacillus species as according to the present disclosure, may provide a further synergistic therapeutic effect in the prevention or treatment of hepatic steatosis, NAFLD and/or NASH.

The term “micro-encapsulation” is used to describe the encapsulation of bacteria in a matrix, coating, or membrane, generally a protective matrix or protective membrane. The (average) diameter of the microcapsules may be between 50 nm and 2 mm, preferably between 100 nm and 1 mm. The matrix, coating or membrane is typically comprised of milk, milk protein, and/or a polymer. The purpose of micro-encapsulation, among other possible purposes, may be to protect bacteria and their components against destruction by the surrounding environment, such as the gastrointestinal environment. The micro-encapsulation of bacteria may also support improved incorporation of bacteria into dairy products, food products, pharmaceutical formulations, and/or pharmaceutical compositions. The micro-encapsulation of bacteria may also support the therapeutic effect.

Various materials may be used for the micro-encapsulation of bacteria, such as pea protein, milk, milk protein, whey protein, casein, xanthan gum, alginate, gelatin, chitosan, carboxymethyl cellulose, starch, and/or carrageenan, and combinations thereof. In a preferred embodiment, the Anaerobutyricum soehngenii or relative thereof, Bifidobacterium species, Akkermansia species and/or Lactobacillus species as according to the present disclosure is micro-encapsulated in one or more polymers.

The subject receiving the combination or composition as taught herein may be selected from the group consisting of human being, non-human primate, mouse, rat, dog, cow, and pig. In a preferred embodiment, the subject is a human.

The at least one Anaerobutyricum soehngenii or relative thereof, the at least one Bifidobacterium species, the at least one Akkermansia species and/or the at least one Lactobacillus species as according to the present disclosure may be comprised in the combination or composition in an amount ranging from 10+to 1015 colony-forming units (CFU). For instance, the at least one Anaerobutyricum soehngenii or relative thereof, the at least one Bifidobacterium species, the at least one Akkermansia species and/or the at least one Lactobacillus species may be comprised in the combination in an amount of 10⁶ CFU to 10¹³ CFU, preferably 10⁷ CFU to 10¹² CFU, preferably 10⁸ CFU to 10¹¹ CFU, more preferably 10⁹ CFU to 10¹¹ CFU, e.g., per dose or per ml or per g of formulation or composition.

In one of the embodiments, the at least one Anaerobutyricum soehngenii or relative thereof, the at least one Bifidobacterium species, the at least one Akkermansia species and/or the at least one Lactobacillus species in the combination or composition taught herein may be incorporated in lyophilized form and/or, micro-encapsulated form (reviewed by, for example, Solanki et al., Bio. Med. Res. Int. 2013, Article ID 620719), or any other form preserving the activity and/or viability of the bacterial strain.

In an embodiment, the combination or composition as taught herein may comprise one or more ingredients, which are suitable for promoting survival and/or viability of the bacterium or strain derived therefrom as taught herein during storage and/or during exposure to bile and/or during passage through the GI tract of a mammal (e.g., a human being). Non-limiting examples of such ingredients include an enteric coating, and controlled release agents allowing passage through the stomach. The skilled person knows how to select suitable ingredients for maintaining a bacterium as taught herein viable and functional, i.e., able to carry out intended function(s).

It may be advantageous to add one or more prebiotic ingredients to the combination as taught herein, for example, to supplement the effects (e.g., production of propionic acid/propionate and/or butyric acid/butyrate or a derivative thereof) of the bacterium as taught herein. The prebiotic ingredients may also enhance the activity and/or stimulate the growth of the bacterium, or a strain derived therefrom, as taught herein. A “prebiotic,” as used herein, generally refers to a non-digestible food ingredient that promotes the growth of beneficial microorganisms in the intestines. Prebiotics or prebiotic products consist mainly of fermentable fibres or non-digestible carbohydrates. The fermentation of these fibres by probiotics promotes the production of beneficial end products, such as SCFAs, particularly butyrate. Non-limiting examples of suitable prebiotics include fibres such as inulin, pectin, and resistant starch, as well as cellobiose, maltose, mannose, salicine, trehalose, amygdalin, arabinose, melibiose, sorbitol, rhamnose and/or xylose. The skilled person is well-acquainted with the field of prebiotics and knows how to select ingredients endowed with prebiotic activity.

In addition or alternative to preventing and/or treating hepatic steatosis, NAFLD and/or NASH, the present disclosure may be used for (enhancing) butyric acid and/or butyrate production, preferably in situ, i.e., in the small intestine. Similarly, the combination according to the present disclosure is also capable of decreasing the level of lactate, e.g., in situ, in the small intestine (lactate is known to be an undesired compound in the intestinal tract).

The term “butyrate” or “butyric acid” (also known under the systematic name “butanoic acid”), as used herein, refers to a carboxylic acid with the structural formula CH₃CH₂CH₂COOH. The term may include derivatives thereof, i.e., compounds derived from butyric acid and includes salts and esters of butyric acid, which are known as butyrate or butanoate. Non-limiting examples of butyrate salts include sodium butyrate, calcium butyrate, magnesium butyrate, manganese butyrate, cobalt butyrate, barium butyrate, lithium butyrate, zinc butyrate, potassium butyrate, ferrous butyrate and the like. Non-limiting examples of butyrate esters (i.e., esters of butyric acid) include cellulose acetate butyrate, methyl butyrate, ethyl butyrate, butyl butyrate, pentyl butyrate, and the like.

Without wishing to be bound by any theories, it is believed that the bacterial strain(s) according to the present disclosure, when administered to a human being or when ingested by a human being in an adequate amount, is/are able to survive and at least transiently colonize the gastrointestinal tract of the human being. This colonization may typically enable greater in situ production of butyric acid/butyrate, although other mechanisms cannot be excluded. Increased in situ production may underlie, at least in part, the beneficial effects in the combination as taught herein, e.g., preventing and/or treatment of hepatic steatosis, Nonalcoholic fatty liver disease (NAFLD), and/or nonalcoholic steatohepatitis (NASH).

In an embodiment, the at least one Anaerobutyricum soehngenii or relative thereof, the at least one Bifidobacterium species, the at least one Akkermansia species and/or the at least one Lactobacillus species may be comprised in a food formulation, feed formulation, feed supplement formulation, food supplement formulation or pharmaceutical formulation. At the same time or alternatively, the at least one Anaerobutyricum soehngenii or relative thereof, the at least one Bifidobacterium species, the at least one Akkermansia species and/or the at least one Lactobacillus species may be comprised in a liquid, liquid beverage (including dairy beverage and fermented beverage), yogurt, cheese, gel, gelatine, gelatine capsule, powder, paste, tablet, or a capsule.

The food or food supplement formulation is preferably a dairy product, more preferably a fermented dairy product, most preferably a yogurt or a yogurt drink.

The pharmaceutical formulation may be, for example, a liquid or solid form, more preferably a solid form solid dosage form, e.g., may be a capsule, a tablet, or a powder. Preferably, a pharmaceutical formulation does not relate to pure water or aqueous medium comprising more than 99 wt. % water.

The formulations as taught herein comprising the combination for use according to the present disclosure may further comprise any acceptable carrier that is suitable for keeping the Anaerobutyricum soehngenii or relative thereof, Bifidobacterium species, Akkermansia species and/or Lactobacillus species as according to the present herein viable until consumption by a subject (e.g., human or animal). For instance, non-limiting examples of acceptable carriers that are suitable for this purpose include any of well-known physiological or pharmaceutical carriers, buffers, and excipients. It will be appreciated that the choice for a suitable physiological or pharmaceutical carrier will depend upon the intended mode of administration of the formulations as taught herein (e.g., oral) and the intended form of the formulations (e.g., beverage, yogurt, powder, capsules, and the like). The skilled person knows how to select a physiological or pharmaceutical carrier, which is suitable for the formulations as taught herein.

The at least one Anaerobutyricum soehngenii or relative thereof, the at least one Bifidobacterium species, the at least one Akkermansia species and/or the at least one Lactobacillus species as taught in the present disclosure may be comprised in the composition in an amount ranging from 10⁴ to 10¹⁵ colony-forming units (CFU). For instance, the at least one Anaerobutyricum soehngenii or relative thereof, the at least one Bifidobacterium species, the at least one Akkermansia species and/or the at least one Lactobacillus species may be comprised in the combination in an amount of 10⁶ CFU to 10¹³ CFU, preferably 10⁷ CFU to 10¹² CFU, preferably 10⁸ CFU to 10¹¹ CFU, more preferably 10⁹ CFU to 10¹¹ CFU, e.g., per dose or per ml or per g of formulation or composition. Alternatively, the amount of the at least one Anaerobutyricum soehngenii or relative thereof, the at least one Bifidobacterium species, the at least one Akkermansia species and/or the at least one Lactobacillus species and/or administration frequency is chosen such that it is between, 10⁶ to 10¹³, preferably 10⁷ to 10¹², preferably 10⁸ to 10¹¹, more preferably 10⁹ to 10¹¹, all in CFU per day.

The terms “comprising” or “to comprise” and their conjugations, as used herein, refer to a situation wherein the terms are used in their non-limiting sense to mean that items following the word are included, but items not specifically mentioned are not excluded. It also encompasses the more limiting verb “to consist essentially of” and “to consist of.”

Reference to an element by the indefinite article “a” or “an” does not exclude the possibility that more than one of the elements is present, unless the context clearly requires that there be one and only one of the elements. The indefinite article “a” or “an” thus usually means “at least one.”

The terms “to increase” and “increased level” and the terms “to decrease” and “decreased level” refer to the ability to significantly increase or significantly decrease or to a significantly increased level or significantly decreased level. Generally, a level is increased or decreased when it is at least 5%, such as 10%, 15%, 20%, 25%, 30%, 35%, 40%, 45%, 50% higher or lower, respectively, than the corresponding level in a control or reference. Alternatively, a level in a sample may be increased or decreased when it is statistically significantly increased or decreased compared to a level in a control or reference.

As used herein, the term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER; Gribskov, M. and Devereux, J., eds., M. Stockton Press, New York, 1991). While there exist a number of methods to measure identity between two polynucleotide or polypeptide sequences, the term “identity” is well known to skilled artisans (Carillo, H., and Lipton, D., SIAM J. Applied Math. (1988) 48:1073). Methods commonly employed to determine identity or similarity between two sequences include, but are not limited to, those disclosed in GUIDE TO HUGE COMPUTERS, Martin J. Bishop, ed., Academic Press, San Diego, 1994, and Carillo, H., and Lipton, D., SIAM J. Applied Math. (1988) 48:1073. Methods to determine identity and similarity are codified in computer programs. For example, NCBI Nucleotide Blast with standard settings (blastn, https://blast.ncbi.nlm.nih.gov/). Preferred computer program methods to determine identity and similarity between two sequences include, but are not limited to, GCS program package (Devereux, J., et al., Nucleic Acids Research (1984) 12(1):387), BLASTP, BLASTN, FASTA (Atschul, S. F. et al., J. Molec. Biol. (1990) 215:403).

As an illustration, by a nucleotide sequence having at least, for example, 95% “identity” to a reference nucleotide sequence, it is intended that the nucleotide sequence is identical to the reference sequence except that there may be up to five-point mutations per each 100 nucleotides of the reference polypeptide sequence. In other words, to obtain a nucleotide sequence being at least 95% identical to a reference nucleotide sequence, up to 5% of the nucleotides in the reference sequence may be deleted and/or substituted with another nucleotide, and/or a number of nucleotides up to 5% of the total nucleotides in the reference sequence may be inserted into the reference sequence. In a sequence listing, a “n” may denote a, t, g, or c.

Should there be an inconsistency between the sequences disclosed in the description and the sequences disclosed in the sequence listing, the sequences disclosed in the description are preferred. Alternatively, the sequences of the sequence listing may be used.

### Experimental Example 1

It has been shown that A. soehngenii can exert effect on glucose metabolism and insulin resistance in the small intestine. In an in vitro model of the Ileum in the presence of a synthetic microbiota A. soehngenii contributes only limited to SCFA production. An experiment was performed to see if this SCFA production could be enhanced by supplementation with the commercially available probiotic Bifidobacterium animalis subsp lactis BLC1 (Bottacini et al. 2011, J. Bacteriol. 193:6387-6388).

Briefly, a synthetic consortium of bacteria was stabilized for 14 days in an Ileum-M-SHIME model (Simulator of Human Intestinal Microbial Ecosystem) comprising the following upper intestinal bacteria with supporting substrates: Lactobacillus spp., Streptococcus spp., Enterococcus spp., Clostridium nexile, Faecalibacterium prausnitzii, Veillonella spp., Prevotella melaninogenica, and Blautia obeum.

A total of 7 ml of this stabilized consortium was seeded with either A. soehngenii; or a combination of A. soehngenii and B. infantis and incubated under anaerobic conditions in the presence of 3 mM bile salts at 37 C. The initial pH of the medium was 7.5.

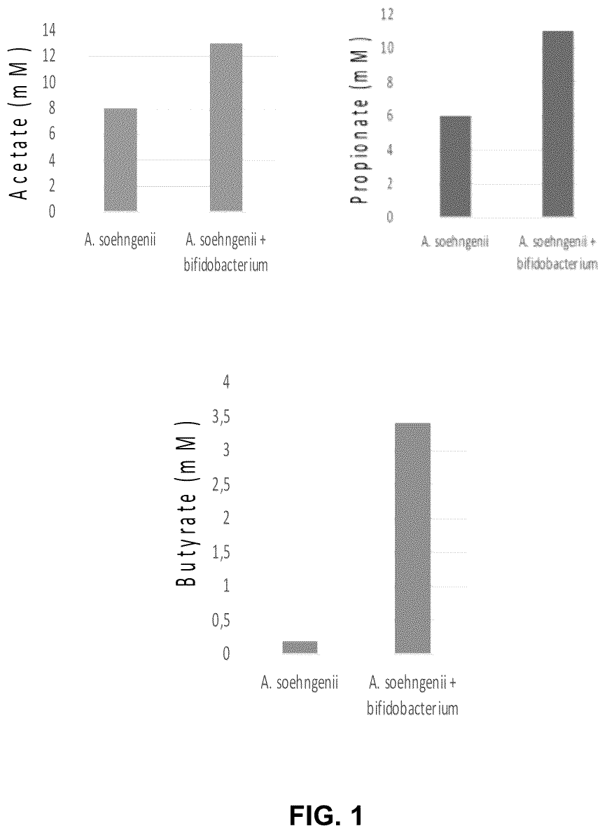

Samples were taken and analyzed for SCFA (acetate, propionate and butyrate) after 24 hours. The result showed a clear increase of all SCFA in the presence of both A. soehngenii and B. infantis compared to the level of SCFA in the presence of only A. soehngenii (FIG. 1).

This demonstrates the metabolic synergy between A. soehngenii and B. infantis under conditions of the upper intestinal tract.

### Experimental Example 2

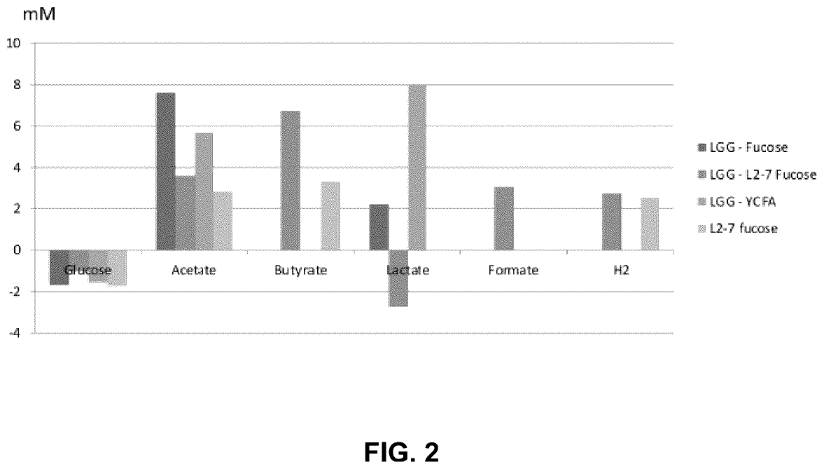

Similarly, the synergy between A. soehngenii L2-7 and various Lactobacillus spp. was shown in incubations with various carbon sources. The combination of A. soehngenii with the commercial probiotic strain Lactobacillus rhamnosus GG (Kankainen et al. 2009106:17193-8) showed a clear synergy during growth on fucose, a common sugar present in the intestinal tract: A. soehngenii does not utilize fucose but L. rhamnosus GG converts fucose into lactate and acetate while the combination of both strains showed conversion of fucose into butyrate, the major metabolic end product of A. soehngenii. See FIG. 2.

### Experimental Example 3

For a period of 20 weeks, two groups of 10 C57BL6/J mice each were placed on a Western diet enriched with 15% fructose in the drinking water (WDF). A control group of 10 mice was placed on a chow diet for the same duration. WDF yielded a diet-induced obesity mouse model (body weight 25% higher than control mice) of non-alcoholic steatohepatitis. From week 12, the DIO-NASH mice were treated with weekly oral gavages of 10\hat{ } CFUs of A. soehngenii or with placebo. At week 20, mice were killed and blood including portal vein sample, as well as liver and gut samples were collected. The DIO-NASH model induced by WDF worked well in inducing NASH: at week 20 average histological steatosis grade was 3, average NAS score 4 and average fibrosis grade was 1 (pericentral or periportal fibrosis).

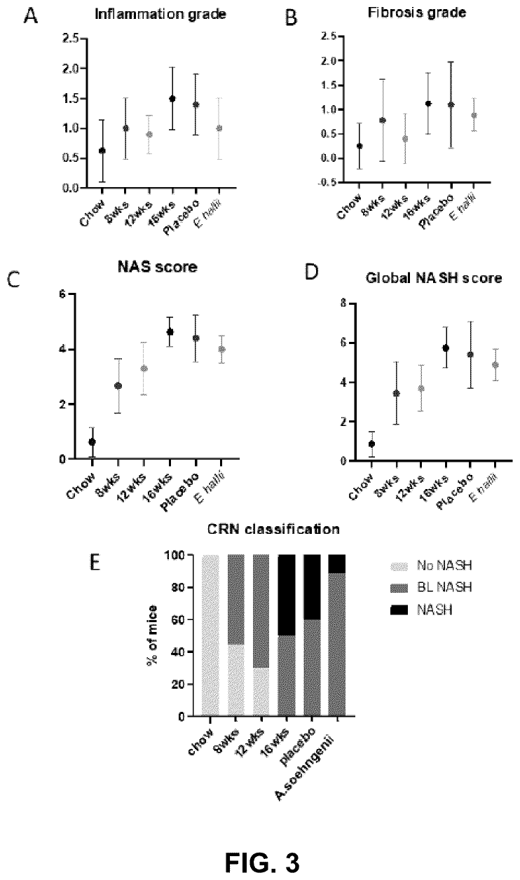

Upon administration of A. soehngenii a clear reduction in inflammation grade, fibrosis grade, NAS score or global NASH score was observed compared to the placebo. Moreover, the number of mice that showed NASH were reduced as compared to the placebo (FIG. 3)

### Experimental Example 4

It was found that co-administration of Anaerobutyricum soehngenii or Anaerobutyricum hallii with a Bifidobacterium species, Akkermansia species and or Lactobacillus species has a beneficial and synergistic effect in patients having or at risk of acquiring hepatic steatosis.

### Methods

### Participants

Caucasian, treatment-naïve, omnivorous individuals with hepatic steatosis on ultrasound are included. The main inclusion criteria are age 21-69 years, male or postmenopausal female, body mass index (BMI) >25 kg/m2 with hepatic steatosis on previous ultrasound with suspicion of NAFLD (based on elevated liver enzymes, impaired glucose tolerance, and severity of steatosis on ultrasound). Exclusion criteria are any history of cardiovascular disease, T2DM, renal disease, cholecystectomy, or compromised immunity; use of proton-pump inhibitors, antibiotics, or anticoagulants in the past 3 months; any current use of medication; a history of moderate to heavy alcohol use (>12 g per day); or other causes of liver disease besides NAFLD (e.g., hemochromatosis, auto-immune hepatitis, cirrhosis, hepatitis B or C, hemochromatosis, alpha-1 antitrypsin deficiency, alcoholic liver disease).

### Intervention

Subjects are treated for at least 24 weeks according to the single or combinatorial treatment arms shown in Table 1. The hepatic necroinflammatory activity score (NAFLD activity score) is measured at baseline and after treatment. Microbiota treatment is given in capsule form, at 10¹⁰ living units per capsule, once daily.

### Liver Biopsy

Percutaneous liver biopsies are performed on the basis of clinical indications according to local standard procedure. All histologic specimens are scored by a liver pathologist who was blinded to any other results. The NASH Clinical Research Network (NASH-CRN) classification (Kleiner et al., Volume 41, Issue 6 June 2005) is assessed with use of hematoxylin and eosin-stained slides for steatosis, inflammation and ballooning, and with a sirius red-stained slide for evaluation of fibrosis. The necroinflammatory activity score (NAS) is determined as described herein.

### Plasma Measurement

Bile acid plasma level is determined by liquid chromatography tandem mass spectrometry (LC-MS/MS).

### Results

As shown, it was determined that the therapeutic effect of Anaerobutyricum soehngenii or Anaerobutyricum hallii increased when administered alone, or when administered in combination with a Bifidobacterium species, Akkermansia species and or Lactobacillus species.

Anaerobutyricum soehngenii or Anaerobutyricum hallii alone has limited ability to improve necroinflammatory activity score. Nonetheless, the Anaerobutyricum soehngenii or Anaerobutyricum hallii alone leads to increased plasma levels of primary bile acids (cholic acid and chenodeoxycholic acid) as well as secondary bile acids (deoxycholic acid and lithocholic acid). These increased plasma levels of bile acids activate Farnesoid-X-Receptor (FXR) and G protein-coupled bile acid receptor GPBARI (TGR5) that lead to increased secretion of GLP-1, which reduces lipogenesis in the liver and reduces liver inflammation (Chiang, Liver Res. 2017 June; 1(1): 3-9).

The effect on bile acid plasma level and efficacy in reduction of the necroinflammatory activity score following treatment is shown in Table 1 accordingly to the following ranking system, wherein the first rank describes the lowest effect and the last rank describes the highest effect: “non-measurable,” “very low,” “low,” “low/medium,” “medium,” “high,” “very high.” In healthy subjects, a lower necroinflammatory activity score can prevent onset of hepatic steatosis, NAFLD and/or NASH. It is expected that results similar to the putative effects as shown in Table 1 can be obtained with larger patient cohorts.

TABLE 1
treatment scheme and effect on bile acid plasma level / lowered necroinflammatory activity score (NAS)

| | | Bifidobacterium | | | |
|---|---|---|---|---|---|
| | | animalis | Bifidobacterium | Bifidobacterium | Bifidobacterium |
| Bacterium | Placebo | subspecies lactis | breve | longum | bifidum |
| Placebo | No change/ | No change/ | No change/ | No change/ | No change/ |
| | Non-measurable | Non-measurable | Non-measurable | Non-measurable | Non-measurable |
| [symbol] | Medium increase | Very high increase | Very high increase | Very high increase | Very high increase |
| [symbol] | in bile acids/ | in bile acids/ | in bile acids/ | in bile acids/ | in bile acids/ |
| [symbol] | Medium effect | Very high effect | Very high effect | Very high effect | Very high effect |
| | on NAS | on NAS | on NAS | on NAS | on NAS |
| [symbol] | Medium increase | Very high increase | Very high increase | Very high increase | Very high increase |
| [symbol] | in bile acids/ | in bile acids/ | in bile acids/ | in bile acids/ | in bile acids/ |
| | Medium effect | Very high effect | Very high effect | Very high effect | Very high effect |
| | on NAS | on NAS | on NAS | on NAS | on NAS |
| [symbol] | Low increase | High increase | High increase | High increase | High increase |
| [symbol] | in bile acids/ | in bile acids/ | in bile acids/ | in bile acids/ | in bile acids/ |
| [symbol] | Low effect | High effect | High effect | High effect | High effect |
| [symbol] | on NAS | on NAS | on NAS | on NAS | on NAS |
| [symbol] | | | | | |
| and [symbol] | | | | | |
| [symbol] | | | | | |

| | Akker [symbol] | Lactobacillus | Lactobacillus | Lactobacillus | Lactobacillus |
|---|---|---|---|---|---|
| Bacterium | [symbol] | [symbol] | [symbol] | acidophilus | [symbol] |
| Placebo | Slight increase/Very | No change/ | No change/ | No change/ | No change/ |
| | low effect on NAS | Non-measurable | Non-measurable | Non-measurable | Non-measurable |
| [symbol] | Very high increase | High increase | High increase | High increase | High increase |
| [symbol] | in bile acids/ | in bile acids/ | in bile acids/ | in bile acids/ | in bile acids/ |
| [symbol] | Very high effect | High effect | High effect | High effect | High effect |
| | on NAS | on NAS | on NAS | on NAS | on NAS |
| [symbol] | Very high increase | High increase | High increase | High increase | High increase |
| [symbol] | in bile acids/ | in bile acids/ | in bile acids/ | in bile acids/ | in bile acids/ |
| | Very high effect | High effect | High effect | High effect | High effect |
| | on NAS | on NAS | on NAS | on NAS | on NAS |
| [symbol] | High increase | Medium increase | Medium increase | Medium increase | Medium increase |
| [symbol] | in bile acids/ | in bile acids/ | in bile acids/ | in bile acids/ | in bile acids/ |
| [symbol] | High effect | Medium effect | Medium effect | Medium effect | Medium effect |
| [symbol] | on NAS | on NAS | on NAS | on NAS | on NAS |
| [symbol] | | | | | |
| and [symbol] | | | | | |
| [symbol] | | | | | |

[symbol]

indicates data missing or illegible when filed

### Experimental Example 5

### Micro-Encapsulation

As shown in this experiment, the effect of non-micro-encapsulated bacteria is compared with the effect of micro-encapsulated bacteria.

The same inclusion criteria of subjects and measurements are used as described in Experimental Example 4. The same ranking system is used as described in Experimental Example 4 to show the efficacy. The applied dose of bacteria is 100-fold lower as compared to Experimental Example 1 to exemplify the effect of bacterial micro-encapsulation. The bacteria are given in capsule form, at 10⁸ living units per capsule once daily.

### Results

Results are shown in Table 2.

TABLE 2
treatment scheme

| Supplement | Effect on necro |
|---|---|
| Bacterium | inflammatory score |
| Placebo | Non-measurable |
| Anaerobutyricum soehngenii | Low |
| Anaerobutyricum soehngenii | Low/medium |
| micro-encapsulated | |
| Anaerobutyricum soehngenii with | Low/medium |
| Bifidobacterium animalis | |
| subspecies lactis | |
| Anaerobuty-ricum soehngenii with | High |
| Bifidobacterium animalis | |
| subspecies lactis micro-encapsulated | |
| Anaerobutyricum soehngenii with | Low/medium |
| Akkermansia muciniphila | |
| Anaerobutyricum soehngenii with | High |
| Akkermansia muciniphila | |
| micro-encapsulated | |

It is expected that similar effects as shown in Table 2 are also obtained with larger patient cohorts.

1-16. (canceled)

17. A method of preventing and/or treating hepatic steatosis in a subject, the method comprising:
administering to the subject Anaerobutyricum soehngenii or a relative thereof having a 16S rRNA gene sequence with at least 97% sequence identity with SEQ ID NO:1 or SEQ ID NO:2 so as to prevent and/or treat hepatic steatosis in the subject, wherein the A. soehngenii or relative thereof is combined with at least one Bifidobacterium species.

18. The method according to claim 17, wherein the at least one Bifidobacterium species is chosen from:
Bifidobacterium animalis subspecies lactis or a relative thereof having a 16S rRNA gene sequence with at least 97% sequence identity with SEQ ID NO:3; and/or
Bifidobacterium breve or a relative thereof having a 16S rRNA gene sequence with at least 97% sequence identity with SEQ ID NO:6.

19. The method according to claim 17, which reduces a hepatic necroinflammatory activity score for the subject.

20. The method according to claim 17, wherein the hepatic steatosis is nonalcoholic fatty liver disease (NAFLD) and/or nonalcoholic steatohepatitis (NASH).

21. The method according to claim 17, wherein the A. soehngenii or relative thereof is combined with at least one Bifidobacterium species and with at least one Akkermansia species.

22. The method according to claim 21, wherein the at least one Akkermansia species has been subjected to pasteurization.

23. The method according to claim 21, wherein the at least one Akkermansia species is Akkermansia muciniphila or a relative thereof having a 16S rRNA sequence with at least 97% sequence identity with SEQ ID NO:12.

24. The method according to claim 17, wherein the A. soehngenii or relative thereof is combined with at least one Bifidobacterium species and with at least one Lactobacillus species.

25. The method according to claim 24, wherein the at least one Lactobacillus species is chosen from:
Lactobacillus acidophilus or a relative thereof having a 16S rRNA sequence with at least 97% sequence identity with SEQ ID NO:14;
Lactobacillus casei or a relative thereof having a 16S rRNA sequence with at least 97% sequence identity with SEQ ID NO:15;
Lactobacillus reuteri or a relative thereof having a 16S rRNA sequence with at least 97% sequence identity with SEQ ID NO: 16; and/or
Lactobacillus rhamnosus or a relative thereof having a 16S IRNA sequence with at least 97% sequence identity with SEQ ID NO:17.

26. The method according to claim 17, which is comprised in fecal matter.

27. The method according to claim 26, wherein the fecal matter is obtained from a donor following a vegan diet.

28. The method according to claim 26, wherein at least 10⁸ cells of the A. soehngenii or relative thereof are comprised in the fecal matter.

29. The method according to claim 17, wherein the A. soehngenii or relative thereof is administered in a micro-encapsulated or lyophilized form.

30. The method according to claim 17, wherein the A. soehngenii or relative thereof is administered in a composition comprising a physiologically acceptable carrier.

31. The method according to claim 30, wherein the A. soehngenii or relative thereof is present in the composition in an amount ranging from 10⁴ to 10¹⁵ colony forming units (CFU).

32. The method according to claim 30, wherein the composition is a pharmaceutical composition and/or food composition.
